SEQUENCE LISTING (11 sequences)

SEQ ID NO: 1 - protein, 442 aa, Homo sapiens [sequence omitted]

SEQ ID NO: 2 - protein, 441 aa, Homo sapiens
  source 1..441: note = amino acid sequence of human CRBN isoform 2
MAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 3 - DNA, 1329 bp, Homo sapiens
  source 1..1329: mol_type = other DNA; note = cDNA sequence encoding human CRBN isoform 1
atggccggcgaaggagatcagcaggacgctgcgcacaacatgggcaaccacctgccgctcctgcctgcagagagtgaggaagaagatgaaatggaagttgaagaccaggatagtaaagaagccaaaaaaccaaacatcataaattttgacaccagtctgccgacatcacatacatacctaggtgctgatatggaagaatttcatggcaggactttgcacgatgacgacagctgtcaggtgattccagttcttccacaagtgatgatgatcctgattcccggacagacattacctcttcagctttttcaccctcaagaagtcagtatggtgcggaatttaattcagaaagatagaacctttgctgttcttgcatacagcaatgtacaggaaagggaagcacagtttggaacaacagcagagatatatgcctatcgagaagaacaggattttggaattgagatagtgaaagtgaaagcaattggaagacaaaggttcaaagtccttgagctaagaacacagtcagatggaatccagcaagctaaagtgcaaattcttcccgaatgtgtgttgccttcaaccatgtctgcagttcaattagaatccctcaataagtgccagatatttccttcaaaacctgtctcaagagaagaccaatgttcatataaatggtggcagaaataccagaagagaaagtttcattgtgcaaatctaacttcatggcctcgctggctgtattccttatatgatgctgagaccttaatggacagaatcaagaaacagctacgtgaatgggatgaaaatctaaaagatgattctcttccttcaaatccaatagatttttcttacagagtagctgcttgtcttcctattgatgatgtattgagaattcagctccttaaaattggcagtgctatccagcgacttcgctgtgaattagacattatgaataaatgtacttccctttgctgtaaacaatgtcaagaaacagaaataacaaccaaaaatgaaatattcagtttatccttatgtgggccgatggcagcttatgtgaatcctcatggatatgtgcatgagacacttactgtgtataaggcttgcaacttgaatctgataggccggccttctacagaacacagctggtttcctgggtatgcctggactgttgcccagtgtaagatctgtgcaagccatattggatggaagtttacggccaccaaaaaagacatgtcacctcaaaaattttggggcttaacgcgatctgctctgttgcccacgatcccagacactgaagatgaaataagtccagacaaagtaatactttgcttgtaa

SEQ ID NO: 4 - DNA, 1326 bp, Homo sapiens
  source 1..1326: mol_type = other DNA; note = cDNA sequence encoding human CRBN isoform 2
atggccggcgaaggagatcagcaggacgctgcgcacaacatgggcaaccacctgccgctcctgcctgagagtgaggaagaagatgaaatggaagttgaagaccaggatagtaaagaagccaaaaaaccaaacatcataaattttgacaccagtctgccgacatcacatacatacctaggtgctgatatggaagaatttcatggcaggactttgcacgatgacgacagctgtcaggtgattccagttcttccacaagtgatgatgatcctgattcccggacagacattacctcttcagctttttcaccctcaagaagtcagtatggtgcggaatttaattcagaaagatagaacctttgctgttcttgcatacagcaatgtacaggaaagggaagcacagtttggaacaacagcagagatatatgcctatcgagaagaacaggattttggaattgagatagtgaaagtgaaagcaattggaagacaaaggttcaaagtccttgagctaagaacacagtcagatggaatccagcaagctaaagtgcaaattcttcccgaatgtgtgttgccttcaaccatgtctgcagttcaattagaatccctcaataagtgccagatatttccttcaaaacctgtctcaagagaagaccaatgttcatataaatggtggcagaaataccagaagagaaagtttcattgtgcaaatctaacttcatggcctcgctggctgtattccttatatgatgctgagaccttaatggacagaatcaagaaacagctacgtgaatgggatgaaaatctaaaagatgattctcttccttcaaatccaatagatttttcttacagagtagctgcttgtcttcctattgatgatgtattgagaattcagctccttaaaattggcagtgctatccagcgacttcgctgtgaattagacattatgaataaatgtacttccctttgctgtaaacaatgtcaagaaacagaaataacaaccaaaaatgaaatattcagtttatccttatgtgggccgatggcagcttatgtgaatcctcatggatatgtgcatgagacacttactgtgtataaggcttgcaacttgaatctgataggccggccttctacagaacacagctggtttcctgggtatgcctggactgttgcccagtgtaagatctgtgcaagccatattggatggaagtttacggccaccaaaaaagacatgtcacctcaaaaattttggggcttaacgcgatctgctctgttgcccacgatcccagacactgaagatgaaataagtccagacaaagtaatactttgcttgtaa

SEQ ID NO: 5 - DNA, 1261 bp, synthetic construct
  source 1..1261: mol_type = other DNA; note = cDNA sequence encoding a fragment from Glu24 to the stop codon of human CRBN isoform 1 or a fragment from Glu23 to the stop codon of human CRBN isoform 2
agagagtgaggaagaagatgaaatggaagttgaagaccaggatagtaaagaagccaaaaaaccaaacatcataaattttgacaccagtctgccgacatcacatacatacctaggtgctgatatggaagaatttcatggcaggactttgcacgatgacgacagctgtcaggtgattccagttcttccacaagtgatgatgatcctgattcccggacagacattacctcttcagctttttcaccctcaagaagtcagtatggtgcggaatttaattcagaaagatagaacctttgctgttcttgcatacagcaatgtacaggaaagggaagcacagtttggaacaacagcagagatatatgcctatcgagaagaacaggattttggaattgagatagtgaaagtgaaagcaattggaagacaaaggttcaaagtccttgagctaagaacacagtcagatggaatccagcaagctaaagtgcaaattcttcccgaatgtgtgttgccttcaaccatgtctgcagttcaattagaatccctcaataagtgccagatatttccttcaaaacctgtctcaagagaagaccaatgttcatataaatggtggcagaaataccagaagagaaagtttcattgtgcaaatctaacttcatggcctcgctggctgtattccttatatgatgctgagaccttaatggacagaatcaagaaacagctacgtgaatgggatgaaaatctaaaagatgattctcttccttcaaatccaatagatttttcttacagagtagctgcttgtcttcctattgatgatgtattgagaattcagctccttaaaattggcagtgctatccagcgacttcgctgtgaattagacattatgaataaatgtacttccctttgctgtaaacaatgtcaagaaacagaaataacaaccaaaaatgaaatattcagtttatccttatgtgggccgatggcagcttatgtgaatcctcatggatatgtgcatgagacacttactgtgtataaggcttgcaacttgaatctgataggccggccttctacagaacacagctggtttcctgggtatgcctggactgttgcccagtgtaagatctgtgcaagccatattggatggaagtttacggccaccaaaaaagacatgtcacctcaaaaattttggggcttaacgcgatctgctctgttgcccacgatcccagacactgaagatgaaataagtccagacaaagtaatactttgcttgtaa

SEQ ID NO: 6 - protein, 467 aa, synthetic construct
  source 1..467: note = Cereblon HDX protein (Figs. 5A & 5C)
MSYYHHHHHHDYDIPTTGLVPRGSMMAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 7 - protein, 836 aa, synthetic construct
  source 1..836: note = DDB1 HDX (Figs. 5B & 5D)
MSYNYVVTAQKPTAVNGCVTGHFTSAEDLNLLIAKNTRLEIYVVTAEGLRPVKEVGMYGKIAVMELFRPKGESKDLLFILTAKYNACILEYKQSGESIDIITRAHGNVQDRIGRPSETGIIGIIDPECRMIGLRLYDGLFKVIPLDRDNKELKAFNIRLEELHVIDVKFLYGCQAPTICFVYQDPQGRHVKTYEVSLREKEFNKGPWKQENVEAEASMVIAVPEPFGGAIIIGQESITYHNGDKYLAIAPPIIKQSTIVCHNRVDPNGSRYLLGDMEGRLFMLLLEKEEQMDGTVTLKDLRVELLGETSIAECLTYLDNGVVFVGSRLGDSQLVKLNVDSNEQGSYVVAMETFTNLGPIVDMCVVDLERQGQGQLVTCSGAFKEGSLRIIRNGIGGNGNSGEIQKLHIRTVPLYESPRKICYQEVSQCFGVLSSRIEVQDTSGGTTALRPSASTQALSSSVSSSKLFSSSTAPHETSFGEEVEVHNLLIIDQHTFEVLHAHQFLQNEYALSLVSCKLGKDPNTYFIVGTAMVYPEEAEPKQGRIVVFQYSDGKLQTVAEKEVKGAVYSMVEFNGKLLASINSTVRLYEWTTEKELRTECNHYNNIMALYLKTKGDFILVGDLMRSVLLLAYKPMEGNFEEIARDFNPNWMSAVEILDDDNFLGAENAFNLFVCQKDSAATTDEERQHLQEVGLFHLGEFVNVFCHGSLVMQNLGETSTPTQGSVLFGTVNGMIGLVTSLSESWYNLLLDMQNRLNKVIKSVGKIEHSFWRSFHTERKTEPATGFIDGDLIESFLDISRPKMQEVVANLQYDDGSGMKREATADDLIKVVEELTRIH

SEQ ID NO: 8 - protein, 59 aa, synthetic construct
  source 1..59: note = ZF 1-2 protein
GSLPNGKLKCDICGIICIGPNVLMVHKRSHTGERPFQCNQCGASFTQKGNLLRHIKLHS

SEQ ID NO: 9 - protein, 59 aa, synthetic construct
  source 1..59: note = ZF 2-3 protein
GSTGERPFQCNQCGASFTQKGNLLRHIKLHSGEKPFKCHLCNYACRRRDALTGHLRTHS

SEQ ID NO: 10 - protein, 87 aa, synthetic construct
  source 1..87: note = ZF 1-2-13 protein
GSLPNGKLKCDICGIICIGPNVLMVHKRSHTGERPFQCNQCGASFTQKGNLLRHIKLHSGEKPFKCHLCNYACRRRDALTGHLRTHS

SEQ ID NO: 11 - protein, 87 aa, synthetic construct; identical to SEQ ID NO: 10
  source 1..87: note = Agnostic ZF 2-3 protein

(19) United States
(12) Patent Application Publication
(10) Pub. No.: US 2025/0052749 A1
(43) Pub. Date: Feb. 13, 2025

(54) SYSTEMS AND METHODS OF SELECTING COMPOUNDS THAT INDUCE CONFORMATIONAL CHANGES IN MUTANT CEREBLON

(71) Applicants: BRISTOL-MYERS SQUIBB COMPANY, Princeton, NJ (US); THE SCRIPPS RESEARCH INSTITUTE, La Jolla, CA (US)

(72) Inventors: Mary E. Matyskiela, San Diego, CA (US); Philip Paul Chamberlain, San Diego, CA (US); Gabriel C. Lander; Edmond R. Watson

(21) Appl. No.: 18/722,850

(22) PCT Filed: Dec. 22, 2022
(86) PCT No.: PCT/US2022/082237

§ 371 Date: Jun. 21, 2024

Related U.S. Application Data
(60) Provisional application No. 63/293,402, filed on Dec. 23, 2021.

(51) Int. Cl. G01N 33/566; G16B 15/30

(52) U.S. Cl. CPC G01N 33/566; G16B 15/30; G01N 2500/04

(57) ABSTRACT

Provided herein are systems and methods for screening and analyzing compounds based upon the elucidation of the interaction among cereblon, its substrates and certain compounds or agents. As an example, a system and method can include a computational model that mimics in silico the cereblon protein. Also provided herein are systems and methods for identifying a compound that induces a conformational change in Cereblon, and in particular P98A mutant cereblon.

### 1. CROSS-REFERENCE TO RELATED APPLICATIONS

This application claims priority to U.S. Provisional Application No. 63/293,402, filed on Dec. 23, 2021, the entirety of which is incorporated herein by reference.

### 2. SEQUENCE LISTING

This application contains a computer readable Sequence Listing which has been submitted in XML file format with this application, the entire content of which is incorporated by reference herein in its entirety. The Sequence Listing XML file submitted with this application is entitled “14247-680-228_SEQ_LISTING.xml”, was created on Dec. 22, 2022, and is 18,988 bytes in size.

### 3. FIELD

This application relates generally processor-implemented systems and methods for analyzing compounds. More specifically, provided herein are screening methods and computational methods based upon the elucidation of the interaction among cereblon, its substrates and certain compounds or agents, including small molecules, peptides, and proteins.

### 4. BACKGROUND

### 4.1 Cereblon

Eukaryotic proteins are targeted for degradation through covalent attachment of ubiquitin moieties to lysine residues on target proteins. Ubiquitination is facilitated by E3 ligases, which coordinate and position targeted substrates, often through adaptor proteins and interchangeable receptor modules. Substrate specificity of one such E3, the Cullin-4 RING ligase (CRL4), is mediated in part by the Cereblon-DDB1 substrate receptor complex. Cereblon has become an important therapeutic target for a class of drugs known as “molecular glues,” which bind to a conserved hydrophobic pocket in Cereblon to create a molecular surface capable of recruiting “neosubstrates” (substrates targeted only in response to drug), leading to their ubiquitination and degradation. Though structural snapshots of Cereblon-DDB1 bound to both drug and substrate have facilitated a molecular description of the stable quaternary complex, the allostery associated with substrate targeting by these evolving ligands remains unknown.

Cereblon is a 50 kDa protein containing three folded domains: an N-terminal Lon protease-like domain (hereafter Lon domain), an intermedial helical bundle (TB), and a C-terminal domain named the Thalidomide-binding domain (TBD) that harbors the IMiD binding pocket. Cereblon functions with Cullin-4 by forming a complex with the adaptor protein DDB1. DDB1 contains three WD-40 beta propeller domains (BPA, BPB, BPC), and the HB of Cereblon docks into a central cleft formed at the interface of BPA and BPC of DDB1. The mobile BPB interacts with Cullin-4, positioning Cereblon-bound substrates for ubiquitination. Over a dozen crystal structures of the Cereblon-DDB1 complex show the Lon domain and TBD of Cereblon tightly interacting with one another, adopting a closed conformation. These structures of the closed conformer bound to small molecules have provided the basis for structure-guided drug design of next-generation molecules with enhanced substrate specificity and potency. While it is thought that association of the TBD and Lon domain in the closed conformer are concomitant with drug and substrate binding, this assumption is challenged by structures of the isolated TBD bound to ligands, demonstrating that the TBD is competent for drug binding in the absence of the Lon domain. Further, two crystal structures have captured the Lon domain and TBD in an open conformation (CRBN^{open}) where they are separated and positioned at an approximately 45° angle relative to one another, raising questions about the assumed constraints associated with drug accessibility and binding, as well as the mechanisms by which molecular glues modify the surface chemistry of Cereblon for substrate recruitment and targeting. See Petzold, G. et al. (2016). Structural basis of lenalidomide-induced CK1α degradation by the CRL4 CRBN ubiquitin ligase. Nature, 532(7597), 127-130; Sievers, Q. L. et al. (2018). Defining the human C2H2 zinc finger degrome targeted by thalidomide analogs through CRBN. Science, 362(6414). If CRBN^{open} represents a physiological conformer, i.e. not an artifact of crystallization as previously suggested, we must reconsider recruitment of drug and substrates in the context of these two conformers, the dynamics of interconversion between the conformers and the effect that molecular glues and substrates have on this process, as well as how these dynamics affect substrate positioning in the context of the full CRL4-Cereblon assembly and proteolytic outcomes.

### 4.2 Immunomodulatory Compounds

Thalidomide, lenalidomide and pomalidomide have shown remarkable responses in patients with multiple myeloma, lymphoma and other hematological diseases such as myelodysplastic syndrome. See Galustian C, et al., Expert Opin Pharmacother., 2009, 10:125-133. These drugs display a broad spectrum of activity, including anti-angiogenic properties, modulation of pro-inflammatory cytokines, co-stimulation of T cells, increased NK cell toxicity, direct anti-tumor effects and modulation of stem cell differentiation.

For example, thalidomide and lenalidomide have emerged as important options for the treatment of multiple myeloma in newly diagnosed patients, in patients with advanced disease who have failed chemotherapy or transplantation, and in patients with relapsed or refractory multiple myeloma. Lenalidomide in combination with dexamethasone has been approved for the treatment of patients with multiple myeloma who have received at least one prior therapy. Pomalidomide may also be administered in combination with dexamethasone. U.S. Patent Publication No. 2004/0029832 A1, the disclosure of which is hereby incorporated in its entirety, discloses the treatment of multiple myeloma.

Another compound provided herein is Compound 1, a cereblon E3 ligase modulator (CELMoD) with approximately 20-fold improved affinity. Compound 1 is the compound (S)-3-(4-((4-(morpholinomethyl)benzyl)oxy)-1-oxoisoindolin-2-yl)piperidine-2,6-dione:

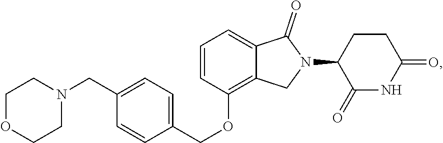 [SMILES: O=C1CC[C@H](N2Cc3c(OCc4ccc(CN5CCOCC5)cc4)cccc3C2=O)C(=O)N1]

or a stereoisomer or mixture of stereoisomers, pharmaceutically acceptable salt, tautomer, prodrug, solvate, hydrate, co-crystal, clathrate, or polymorph thereof. A method for preparing Compound 1 is described in U.S. Pat. No. 8,518,972, which is incorporated herein by reference in its entirety. In one embodiment, Compound 1 is used in the methods provided herein. In one embodiment, a hydrochloride salt of Compound 1 is used in the methods provided herein.

Another compound provided herein is compound 2, a next generation CELMoD developed for the treatment of relapse/refractory multiple myeloma (RRMM). Compound 2 is the compound (S)-4-(4-(4-(((2-(2,6-dioxopiperidin-3-yl)-1-oxoisoindolin-4-yl)oxy)methyl)benzyl)piperazin-1-yl)-3-fluorobenzonitrile:

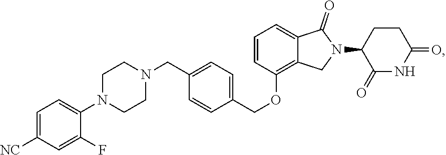 [SMILES: N#Cc1ccc(N2CCN(Cc3ccc(COc4cccc5c4CN([C@H]4CCC(=O)NC4=O)C5=O)cc3)CC2)c(F)c1]

or a stereoisomer or mixture of stereoisomers, pharmaceutically acceptable salt, tautomer, prodrug, solvate, hydrate, co-crystal, clathrate, or polymorph thereof. A method for preparing Compound 2 is described in U.S. Pat. No. 10,357,489, which is incorporated herein by reference in its entirety. In one embodiment, Compound 2 is used in the methods provided herein. In one embodiment, a hydrobromide salt of Compound 2 is used in the methods provided herein.

Understanding the interactions of CRBN, the CRBN E3 ubiquitin-ligase complex, or one or more substrates of CRBN with thalidomide, lenalidomide, pomalidomide and other drug targets will allow the definition of the molecular mechanisms of efficacy and/or toxicity and may lead to drugs with improved efficacy and toxicity profiles.

### 4.3 P98A Muant Cereblon

P98A mutant Cereblon is a mutated version of Cereblon found in human patients, which is linked to relapse and/or refraction to lenalidomide treatment of multiple myeloma. The P98A mutation occurs at the interface between Lon and TBD. The mutant Cereblon protein cannot close with pomalidomide or Ikaros. This serves as impetus for the development of new CELMoDs which have been selected for improved degradation. For example, Compound 2 is improved in the context of mutations like P98A and is used for the treatment of relapse or refractory multiple myeloma (RRMM).

### 5. BRIEF SUMMARY

Cereblon is an E3 ligase adaptor protein that is targeted by anti-cancer immunomodulatory imide (IMiD) drugs and has become an important target of next-generation tumoricidal and immunomodulatory molecules. Described herein is a cryo-EM technique that allows the visualization of dynamic structures and the classification and quantification of their distributions. Using this and other techniques, we are the first to report and understand that Cereblon-DDB1 adopts a semi-rigid open conformation, and that the association of IMiD drugs, such as Pomalidomide, triggers an allosteric switch to the closed conformation. Furthermore, we note that Cereblon with the P98A mutation cannot close with Pomalidomide, but Compound 2 can overcome this mutation. P98A mutant Cereblon can close and recruit Ikaros in In the presence of Compound 2.

As such, describe herein are experimental techniques, Cereblon conformations, and its interactions and responses to drugs. We also describe drug screening and computational methods that exploit these discoveries.

In one aspect, provided herein is a method of identifying a compound that induces a conformational change in P98A mutant Cereblon (P98A CRBN), wherein the method comprises: contacting the compound with P98A CRBN; and assessing the P98A CRBN for a conformational change.

In certain embodiments, the P98A CRBN conformational change is indicative of a compound that induces the P98A CRBN conformational change.

In a second aspect, provided herein is a method of identifying a compound that induces a conformational change in P98A mutant Cereblon (P98A CRBN), wherein the method comprises accessing biophysical and dynamics data for P98A CRBN; analyzing the biophysical and dynamics data to identify one or more potential allosteric sites on P98A CRBN; computationally screening a plurality of chemical compounds to determine a binding energy between each of the subset of allosteric sites and each of the plurality of chemical compounds; computationally modeling the effect of the chemical compounds binding to each of the plurality of allosteric sites; quantifying each of a plurality of conformations of P98A CRBN while bound to the chemical compounds and while not bound to the compounds; ranking the compounds based on achieving a conformation change in P98A CRBN; and selecting a subset of top ranking chemical compounds comprising a potential compound that induced a conformational change in P98A CRBN.

In a third aspect, provided herein is a method of identifying a compound that induces a conformational change in P98A mutant Cereblon (P98A CRBN), wherein the method comprises: (a) using structural information describing the structure of P98A CRBN; (b) performing a molecular dynamics (MD) simulation of the structure; (c) using a clustering algorithm to identify dominant conformations of the structure from the MD simulation; (d) selecting the dominant conformations of the structure identified from the clustering algorithm; (e) providing structural information describing conformers of one or more compounds; (f) using a docking algorithm to dock the conformers of the one or more compounds of step (e) to the dominant conformations of step (d); (g) identifying a plurality of preferred binding conformations for each of the combinations of protein and compound; (h) optimizing the preferred binding conformations using scalable MD; and (i) determining if the compound causes a conformation change in P98A CRBN in the preferred binding conformations.

In some embodiments, if the compound causes a conformational change in the preferred binding conformations, the compound is identified. In some embodiments, if the compound does not causes a conformational change in the preferred binding conformations, the compound is not identified.

In some embodiments, said steps (a) through (i) are executed on one or more processors.

### 6. BRIEF DESCRIPTION OF THE DRAWINGS

FIGS. 1A-D depict that CRBN^{open} is allosterically modulated to CRBN^{closed} by Pomalidomide.

FIG. 1A is a surface representation of an approximately 4 Å resolution cryo-EM reconstruction of Cereblon/DDB1 in the unliganded apo form.

FIG. 1B is a ribbon representation of Cereblon-DDB1 protein modelled from density.

FIG. 1C is a surface representation of an approximately 4 Å resolution cryo-EM reconstruction of Cereblon-DDB1 in the closed form in complex with Pomalidomide.

FIG. 1D is a ribbon representation of Cereblon-DDB1^{ΔBPB}.

FIGS. 2A-C depict the structural ensemble of Cereblon-DDB1.

FIG. 2A is an 3.5 Å cryo-EM consensus refinement of Cereblon-DDB1 isolated in the apo state.

FIG. 2B shows an image of Gaussian filtered Cereblon-DDB1, highlighting the details of the dynamic region of Thalidomide Binding Domain (TBD).

FIG. 2C shows the three distinct locations of DDB1 BPB that are termed linear, hinged, and state 3.

FIG. 3 depicts that Cereblon adopts open conformation with DDB1^{ΔBPB}.

FIGS. 4A-C depict that CELMoDs more substantially impact Cereblon and recruit Ikaros to CRBN^{closed}.

FIG. 4A shows a surface representation of an approximately 4 Å resolution cryo-EM structure of Pomalidomide-induced CRBN^{closed} (upper right), and an approximately 3.8 Å resolution cryo-EM density from Compound 1-induced CRBN^{closed} (bottom right).

FIG. 4B shows space-filling representation of Cereblon models with residue-specific shading according to changes in solvency upon addition of Compound 1 as detected by HDX-MS.

FIG. 4C is a composite map of local refinements for Ikaros/Cereblon/DDB1.

FIGS. 5A-D depict the results of hydrogen-deuterium exchange experiments.

FIG. 5A shows a per-residue peptide mapping of Cereblon HDX differential upon addition of Compound 1 to Cereblon-DDB1.

FIG. 5B shows a per-residue peptide mapping of DDB1 HDX differential upon addition of Compound 1 to Cereblon-DDB1.

FIG. 5C shows a per-residue peptide mapping of Cereblon HDX differential upon addition of Compound 1 and Ikaros ZF1-2-3 to Cereblon-DDB1.

FIG. 5D shows a per-residue peptide mapping of DDB1 HDX differential upon addition of Compound 1 and Ikaros ZF1-2-3 to Cereblon-DDB1.

FIG. 6A-C depict that Pomalidomide is recruited to both CRBN^{open} and CRBN^{closed} but recruits only a single ZF to CRBN^{closed}.

FIG. 6A shows zoomed-in views of TBD from low-resolution cryo-EM reconstructions of unliganded apo CRBN^{open} and Pomalidomide-bound CRBN^{open}.

FIG. 6B is a sequence alignment for Ikaros proteins.

FIG. 6C is an approximately 4 Å cryo-EM reconstructions of Cereblon-DDB1^{ΔBPB} bound to different versions of Ikaros tandem ZF protein.

FIGS. 7A-B depict Ikaros recruitment.

FIG. 7A is a 3.4 Å sharpened map of Cereblon-DDB1 bound to Ikaros ZF 2-3.

FIG. 7B is an unsharpened image of FIG. 7A.

FIGS. 8A-B depict that DDB1 and next-generation CELMoDs further poise Cereblon substrates for ubiquitination in disease contexts.

FIG. 8A show approximately 3.6 Å resolution cryo-EM reconstructions of Cereblon-DDB1 with BPB in linear (left), hinged (middle) or twisted (right) positions.

FIG. 8B is an approximately 4 Å resolution cryo-EM reconstruction of P98A Cereblon-DDB1 complexed with Compound 2 and Ikaros ZF1-2-3.

FIG. 8C is a cartoon model summarizing mechanism and impact of IMiD- and CELMoD-induced allosteric regulation of Cereblon.

FIG. 9 depict that Compound 2 overcomes P98A.

FIGS. 10A-B depict that Pomalidomide recruits Ikaros ZF to CRBN^{closed}.

FIG. 11A shows an approximately 2.8 Å model of DDB1^{ΔBPB}.

FIG. 11B shows that Cereblon in the apo state adopts an open form.

FIG. 11C shows the structural details of the interactions between Cereblon and DDB1.

FIG. 12 depicts P98A mutant Cereblon in the open and closed conformations.

FIGS. 13-15 depicts methods of identifying a compound that induces a conformational change in P98A mutant Cereblon (P98A CRBN).

FIG. 16 depicts an example block diagram depicting an environment wherein users can interact with a grid computing environment.

FIG. 17 depicts an example block diagram depicting hardware and software components for the grid computing environment.

FIG. 18 depicts example schematics of data structures utilized by a compound-selection system.

FIG. 19 depicts an example block diagram depicting a compound-selection system provided on a stand-alone computer for access by a user.

FIG. 20 depicts a typical processing workflow described for Cereblon/DDB1 in the unliganded apo form.

### 7. DETAILED DESCRIPTION

### 7.1 Definitions

Unless defined otherwise, all technical and scientific terms used herein have the same meaning as is commonly understood by one of ordinary skill in the art. In the event that there is a plurality of definitions for a term herein, those in this section prevail unless stated otherwise.

As used herein, the terms “about” and “approximately” mean within 20%, preferably within 10%, and more preferably within 5% (or 1% or less) of a given value or range.

As used herein, the terms “assaying,” “assessing,” “determining,” “evaluating,” and “measuring” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

As used herein, the term “binding conformations” refers to the orientation of a ligand to a receptor when bound or docked to each other.

As used herein, the term “binding energies” is understood to mean the “free energy of binding” (ΔG^{º}) of a ligand to a receptor. Under equilibrium conditions, this binding energy is equal to ΔG^{º}=ΔH^{º}−T ΔS⁵⁸⁸=−R T Log(K_{eq}), where the symbols have their customary meanings. In certain embodiments, the methods disclosed herein allow calculation of binding energies for various ligand-receptor complexes, for example, various compounds bound to CRBN.

The terms “cereblon” or “CRBN” and similar terms refers to the polypeptides (“polypeptides,” “peptides” and “proteins” are used interchangeably herein) comprising the amino acid sequence any CRBN, such as a human CRBN protein (e.g., human CRBN isoform 1, GenBank Accession No. NP_057386; or human CRBN isoforms 2, GenBank Accession No. NP_001166953, each of which is herein incorporated by reference in its entirety), and related polypeptides, including SNP variants thereof. Related CRBN polypeptides include allelic variants (e.g., SNP variants); splice variants; fragments; derivatives; substitution, deletion, and insertion variants; fusion polypeptides; and interspecies homologs, which, in certain embodiments, retain CRBN activity and/or are sufficient to generate an anti-CRBN immune response.

As used herein, the term “clustering algorithm,” when applied to a trajectory of the MD simulations disclosed herein, refers to computational approaches for grouping similar conformations in the trajectory into clusters.

As used herein, the term “compound,” “drug,” and “ligand” are used interchangeably, and refer to any small molecule which is capable of binding to a target receptor, such as CRBN.

As used herein, the terms “computer memory” and “computer memory device” refer to any storage media readable by a computer processor. Examples of computer memory include, but are not limited to, RAM, ROM, computer chips, digital video discs (DVD), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the term “dock” or “docking” refers to using a model of a ligand and receptor to simulate association of the ligand-receptor at a proximity sufficient for at least one atom of the ligand to be within bonding distance of at least one atom of the receptor. The term is intended to be consistent with its use in the art pertaining to molecular modeling. A model included in the term can be any of a variety of known representations of a molecule including, for example, a graphical representation of its three-dimensional structure, a set of coordinates, set of distance constraints, set of bond angle constraints or set of other physical or chemical properties or combinations thereof. In certain embodiments, the ligand is a compound, for example a small molecule, and the receptor is a protein macromolecule, for example, CRBN.

As used herein, the term “docking algorithm” refers to computational approaches for predicting the energetically preferred orientation of a ligand to a receptor when bound or docked to each other to form a stable ligand-receptor complex. Knowledge of the preferred orientation in turn may be used to predict the strength of association or binding affinity between ligand and receptor using, for example, scoring functions. In certain embodiments, the ligand is a compound, for example a small molecule, and the receptor is a protein macromolecule, for example, CRBN.

As used herein, the term “dominant conformation” or “dominant conformations” refers to most highly populated orientation(s) of a ligand to a receptor when bound or docked to each other. In certain embodiments, when applied to the trajectories of the MD simulations disclosed herein, a clustering algorithm is used to determine the “dominant conformation” or “dominant conformations.”

As used herein, the term “drug design” or “rational drug design” refers to methods of processes of discovering new drugs based on the knowledge of a biological target. In certain embodiments of the methods disclosed herein, the biological target is a protein macromolecule, for example, CRBN. Those of ordinary skill in the art will appreciate that drug design that relies on the knowledge of the three-dimensional structure of the biomolecular target is also known as “structure-based drug design.” Those of ordinary skill in the art will also understand that drug design may rely on computer modeling techniques, which type of modeling is often referred to as “computer-aided drug design.”

As used herein, the term “dynamics,” when applied to macromolecule and macromolecular structures, refers to the relative motion of one part of the molecular structure with respect to another. Examples include, but are not limited to: vibrations, rotations, stretches, domain motions, hinge motions, sheer motions, torsion, and the like. Dynamics may also include motions such as translations, rotations, collisions with other molecules, and the like.

As used herein, the term “energy minimization” refers to computational methods for computing stable states of interacting atoms, groups of atoms or molecules, including macromolecules, corresponding to global and local minima on their potential energy surface. Starting from a non-equilibrium molecular geometry, energy minimization employs the mathematical procedure of optimization to move atoms so as to reduce the net forces (the gradients of potential energy) on the atoms until they become negligible.

As used herein, the term “flexible” or “flexibility,” when applied to macromolecule and macromolecular structures defined by structural coordinates, refers to a certain degree of internal motion about these coordinates, e.g., it may allows for bond stretching, rotation, etc.

As used herein, the term “high throughput screening” refers to a method that allows a researcher to quickly conduct chemical, genetic or pharmacological tests, the results of which provide starting points for drug design and for understanding the interaction or role of a particular biochemical process in biology. In certain embodiments, the high throughput screening is through virtual in silico screening, for example, using computer-based methods or computer-based models.

As used herein, the term “immunomodulatory compound,” “immunomodulatory drug,” or “IMiD” refers generally to a molecule or agent capable of altering the immune response in some way. Non-limiting examples of immunomodulatory compounds include those disclosed in Section 2.2 above.

As used herein, the terms “isolated” and “purified” refer to isolation of a substance (such as mRNA, antibody or protein) such that the substance comprises a substantial portion of the sample in which it resides, i.e. greater than the substance is typically found in its natural or un-isolated state. Typically, a substantial portion of the sample comprises, e.g., greater than 1%, greater than 2%, greater than 5%, greater than 10%, greater than 20%, greater than 50%, or more, usually up to about 90%-100% of the sample. For example, a sample of isolated protein can typically comprise at least about 1% total protein. Techniques for purifying proteins are well known in the art and include, for example, size-exclusion and ion-exchange chromatography.

As used herein, the term “molecular dynamics simulation” (MD or MD simulation) refers to computer-based molecular simulation methods in which the time evolution of a set of interacting atoms, groups of atoms or molecules, including macromolecules, is followed by integrating their equations of motion. The atoms or molecules are allowed to interact for a period of time, giving a view of the motion of the atoms or molecules. Thus, the MD simulation may be used to sample conformational space over time to predict the lowest energy, most populated, members of a conformational ensemble. Typically, the trajectories of atoms and molecules are determined by numerically solving the Newton's equations of motion for a system of interacting particles, where forces between the particles and potential energy are defined by molecular mechanics force fields. However, MD simulations incorporating principles of quantum mechanics and hybrid classical-quantum mechanics simulations are also available and may be contemplated herein.

As used herein, the term “scalable molecular dynamics” (scalable MD) refers to computational simulation methods which are suitably efficient and practical when applied to large situations (e.g., a large input data set, a large number of outputs or users, or a large number of participating nodes in the case of a distributed system). In certain embodiments, the methods disclosed herein use scalable MD for simulation of the large systems disclosed herein, for example, CRBN with explicit solvent and ion molecules, free, or bound to ligand.

As used herein, the term “molecular simulation” refers to a computer-based method to predict the functional properties of a system, including, for example, thermodynamic properties, thermochemical properties, spectroscopic properties, mechanical properties, transport properties, and morphological information. In certain embodiments, the molecular simulation is a molecular dynamics (MD) simulation.

As used herein, the term “preferred binding conformation” refers to the energetically preferred orientation of a ligand to a receptor when bound or docked to each other to form a stable ligand-receptor complex.

As used herein, the term “optimized preferred binding conformation” refers to the energetically preferred orientation of a ligand to a receptor when bound or docked to each other to form a stable ligand-receptor complex, following optimizing the preferred binding conformations using MD.

As used herein the terms “polypeptide” and “protein” as used interchangeably herein, refer to a polymer of amino acids of three or more amino acids in a serial array, linked through peptide bonds. The term “polypeptide” includes proteins, protein fragments, protein analogues, oligopeptides and the like. The term polypeptide as used herein can also refer to a peptide. The amino acids making up the polypeptide may be naturally derived, or may be synthetic. The polypeptide can be purified from a biological sample.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “sample” relates to a material or mixture of materials, typically, although not necessarily, in fluid form, containing one or more components of interest.

As used herein, the term “structural information” refers to the three dimensional structural coordinates of the atoms within a macromolecule, for example, a protein macromolecule such as hERG1.

As used herein, the term “three-dimensional (3D) structure” refers to the Cartesian coordinates corresponding to an atom's spatial relationship to other atoms in a macromolecule, for example, a protein macromolecule such as CRBN. Structural coordinates may be obtained using NMR techniques, as known in the art, or using X-ray crystallography as is known in the art. Alternatively, structural coordinates can be derived using molecular replacement analysis or homology modeling. Various software programs allow for the graphical representation of a set of structural coordinates to obtain a three dimensional representation of a molecule or molecular complex.

### 7.2 Computer-Based Embodiments

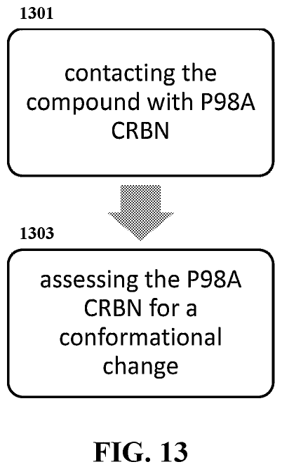

An overview of the methods disclosed herein, including computer-based molecular simulations and molecular models, is provided in FIGS. 13-15.

In one aspect, provided herein is a method of identifying a compound that induces a conformational change in P98A mutant Cereblon (P98A CRBN). FIG. 13 illustrates the method, wherein the method comprises: contacting the compound with P98A CRBN (1301); and assessing the P98A CRBN for a conformational change (1303).

In certain embodiments, the P98A CRBN conformational change in 1303 is indicative of a compound that induces the P98A CRBN conformational change. In certain embodiments, the conformational change in 1303 is a change from an open to closed conformation.

In certain embodiments, the C-terminal Thalidomide Binding Domain (TBD) and LON-like domain (LON) domains of the P98A CRBN are in the open conformation. In some embodiments, the open conformation has a three-dimensional structure having the atomic coordinates set forth in Table 1. In some embodiments, the TBD and LON domains are in the closed conformation. In one embodiment, the closed conformation has a three-dimensional structure having the atomic coordinates set forth in Table 2.

In certain embodiments, the conformational change in 1303 occurs in a cereblon modifying agent (CMA) binding pocket of the P98A CRBN and has an effect on W380, W386 and/or W400 of P98A CRBN (wherein the amino acid numbering correlates to human P98A CRBN). In certain embodiments, the conformational change in 1303 has an effect on E377 of P98A CRBN. In certain embodiments, the conformational change in 1303 has an effect on V388 of P98A CRBN.

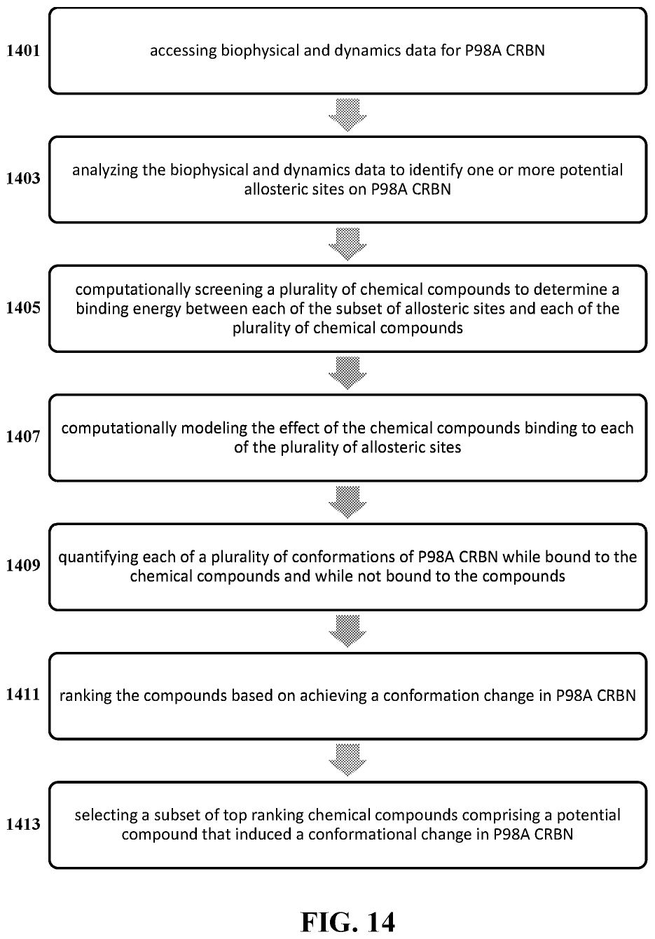

In a second aspect, provided herein is a method of identifying a compound that induces a conformational change in P98A mutant Cereblon (P98A CRBN). FIG. 14 illustrates the method, wherein the method comprises: accessing biophysical and dynamics data for P98A CRBN (1401); analyzing the biophysical and dynamics data to identify one or more potential allosteric sites on P98A CRBN (1403); computationally screening a plurality of chemical compounds to determine a binding energy between each of the subset of allosteric sites and each of the plurality of chemical compounds (1405); computationally modeling the effect of the chemical compounds binding to each of the plurality of allosteric sites (1407); quantifying each of a plurality of conformations of P98A CRBN while bound to the chemical compounds and while not bound to the compounds (1409); ranking the compounds based on achieving a conformation change in P98A CRBN (1411); and selecting a subset of top ranking chemical compounds comprising a potential compound that induced a conformational change in P98A CRBN (1413).

In some embodiments, the biophysical and dynamics data in 1401 and/or 1403 are obtained by one or more of nuclear magnetic resonance (NMR), X-ray crystallography, cryogenic electron microscopy (Cryo-EM), neutron scattering, and hydrogen-deuterium exchange. In some embodiments, the biophysical and dynamics data in 1401 and/or 1403 are obtained by Cryo-EM.

In some embodiments, the conformational change in 1411 and/or 1413 is a change from an open to closed conformation.

In some embodiments, the C-terminal Thalidomide Binding Domain (TBD) and LON-like domain (LON) domains of the P98A CRBN are in the open conformation. In some embodiments, the open conformation has a three-dimensional structure having the atomic coordinates set forth in Table 1. In some embodiments, the TBD and LON domains are in the closed conformation. In some embodiments, the closed conformation has a three-dimensional structure having the atomic coordinates set forth in Table 2.

In some embodiments, the conformational change in 1411 and/or 1413 occurs in a cereblon modifying agent (CMA) binding pocket of the P98A CRBN and has an effect on W380, W386 and/or W400 (wherein the amino acid numbering correlates to human P98A CRBN). In some embodiments, the conformational change in 1411 and/or 1413 has an effect on E377. In some embodiments, the conformational change in 1411 and/or 1413 has an effect on V388 of P98A CRBN.

In some embodiments, the method further comprising computationally modeling a derived set of chemical compounds based on strong binding compounds with different functional groups to improve the binding on the allosteric sites. In some embodiments, the computational modeling is performed using high-throughput computational docking software.

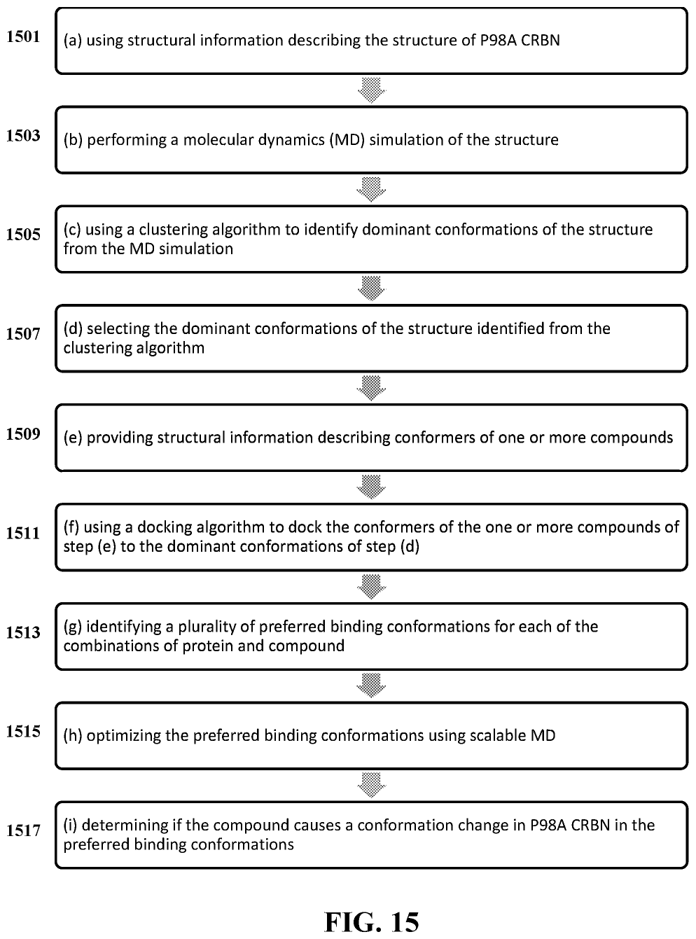

In a third aspect, provided herein is a method of identifying a compound that induces a conformational change in P98A mutant Cereblon (P98A CRBN). FIG. 15 illustrates the method, wherein the method comprises wherein the method comprises: using structural information describing the structure of P98A CRBN (1501); performing a molecular dynamics (MD) simulation of the structure (1503); using a clustering algorithm to identify dominant conformations of the structure from the MD simulation (1507); selecting the dominant conformations of the structure identified from the clustering algorithm (1509); providing structural information describing conformers of one or more compounds (1511); using a docking algorithm to dock the conformers of the one or more compounds of step 1509 to the dominant conformations of step 1507; identifying a plurality of preferred binding conformations for each of the combinations of protein and compound (1513); optimizing the preferred binding conformations using scalable MD (1515); and determining if the compound causes a conformation change in P98A CRBN in the preferred binding conformations (1517).

In some embodiments, if the compound causes a conformational change in the preferred binding conformations in 1517, the compound is identified. In some embodiments, if the compound does not causes a conformational change in the preferred binding conformations in 1517, the compound is not identified.

In some embodiments, the steps 1501 to 1517 are executed on one or more processors.

In some embodiments, the structural information in 1501 and/or 1509 is obtained by one or more of nuclear magnetic resonance (NMR), X-ray crystallography, cryogenic electron microscopy (Cryo-EM), and a homology model. In some embodiments, the structural information is obtained by Cryo-EM.

In some embodiments, the conformational change is a change from an open to closed conformation. In some embodiments, the C-terminal Thalidomide Binding Domain (TBD) and LON-like domain (LON) domains of the P98A CRBN are in the open conformation. In some embodiments, the open conformation has a three-dimensional structure having the atomic coordinates set forth in Table 1. In some embodiments, the TBD and LON domains are in the closed conformation. In some embodiments, the closed conformation has a three-dimensional structure having the atomic coordinates set forth in Table 2.

Individual elements and steps of the methods disclosed herein are now described.

### 7.2.1 Computational Aspects

In certain aspects, provided herein are computational methods for selecting a compound that is likely to induce a conformational change in P98A mutant Cereblon.

In certain embodiments, the computational methods comprise a computational dynamic model. In certain embodiments, the computational dynamic model comprises a molecular simulation that samples conformational space over time. In certain embodiments, the molecular simulation is a molecular dynamics (MD) simulation.

In certain embodiments, the method comprising the steps of: a) using structural information describing the structure of the CRBN protein; b) performing a molecular dynamics (MD) simulation of the protein structure; c) using a clustering algorithm to identify dominant conformations of the protein structure from the MD simulation; d) selecting the dominant conformations of the protein structure identified from the clustering algorithm; e) providing structural information describing conformers of one or more compounds; f) using a docking algorithm to dock the conformers of the one or more compounds of step e) to the dominant conformations of step d); g) identifying a plurality of preferred binding conformations for each of the combinations of protein and compound; h) optimizing the preferred binding conformations using scalable MD; and i) determining if the compound causes a conformation change in P98A CRBN in the preferred binding conformations; wherein one or more of the steps a) through i) are not necessarily executed in the recited order.

In certain embodiments, the structural information of step a) is a three-dimensional (3D) structure. In certain embodiments, the structural information of step a) is an X-ray crystal structure, a cryo-EM structure, an NMR solution structure, or a homology model, as disclosed herein.

In certain embodiments, step e) comprises providing the chemical structure of a compound and determining the conformers of the compound. In certain embodiments, the chemical structure of the compound defines the conformers.

In certain embodiments, one or more of the steps a) through i) of the method are performed in the recited order.

In certain embodiments, steps a) through i) of the method are executed on one or more processors.

### 7.2.2 Structural Information of Cereblon (CRBN) Protein

In certain embodiments, the method comprises the step of using structural information describing the structure of a CRBN protein. In certain embodiments, the CRBN protein is also referred to as a “receptor” or “target” and the terms “protein,” “receptor” and “target” are used interchangeably.

In certain embodiments, the structural information describing the structure of the CRBN protein is from a homology model.

In certain embodiments, the structural information describing the structure of the CRBN protein is from an NMR solution structure. Multidimensional heteronuclear NMR techniques for determination of the structure and dynamics of macromolecules are known to those of ordinary skill in the art.

In certain embodiments, the structural information describing the structure of the CRBN protein is from an X-ray crystal structure. X-ray crystallographic techniques for determination of the structure of macromolecules are also known to those of ordinary skill in the art.

In certain embodiments, the structural information describing the structure of the CRBN protein is from an cryo-EM structure. Cryo-EM techniques for determination of the structure of macromolecules are also known to those of ordinary skill in the art.

### 7.2.3 Structural Information of the Compound (Ligand)

In certain embodiments, the method comprises providing structural information describing conformers of one or more compounds or ligands. As used herein, the terms “compound” and “ligand” are interchangeable.

One of ordinary skill in the art will understand that a chemical compound can adopt differing three-dimensional (3-D) shapes or “conformers” due to rotation of atoms about a bond. Conformers can thus interconvert by rotation around a single bond without breaking. A particular conformer of a ligand may provide a complimentary geometry to a protein, and promote binding.

In certain embodiments, the structural information of describing conformers of one or more compounds or ligands is obtained from the chemical structure of a compound or ligand.

In certain embodiments, the structural information of the compound is based upon an IMiD compound being studied or developed by universities, pharmaceutical companies, or individual inventors. Typically, the compound will be a small organic molecule having a molecular weight under 900 atomic mass units. Structural information of the compound may be provided in 2D or 3D, using ChemDraw or simple structural depictions, or by entry of the compound's chemical name. Computer-based modeling of the compound may be used to translate the chemical name or 2D information of the compound into a 3D representative structure.

The software LigPrep from the Schrödinger package (Schrödinger Release 2013-2: LigPrep, version 2.7, Schrödinger, LLC, New York, NY, 2013) may be used to translate the 2D information of the compound (ligand) into a 3D representative structure which provides the structural information. LigPrep may also be used to generate variants of the same compound (ligand) with different tautomeric, stereochemical, and ionization properties. All generated structures may be conformationally relaxed using energy minimization protocols included in LigPrep.

In certain embodiments, the compound is an anticancer agent.

In certain embodiments, the compound is selected from thalidomide, pomalidomide, lenalidomide, Compound 1, and Compound 2.

### 7.2.4 Energy Minimization

In certain embodiments, the cryo-EM structure, X-ray crystal structure, NMR solution structures, homology models, molecular models, or generated structures disclosed herein are subjected to energy minimization prior to performing an MD simulation.

The goal of energy minimization is to find a local energy minimum for a potential energy function. A potential energy function is a mathematical equation that allows the potential energy of a molecular system to be calculated from its three-dimensional structure. Examples of energy minimization algorithms include, but are not limited to, steepest descent, conjugated gradients, Newton-Raphson, and Adopted Basis Newton-Raphson (Molecular Modeling: Principles and Applications, Author A. R. Leach, Pearson Education Limited/Prentice Hall (Essex, England), 2nd Edition (2001) pages: 253-302). It is possible to use several methods interchangeably.

### 7.2.5 Molecular Simulations

In certain aspects, provided herein are computational methods for selecting a compound that is likely to induce a conformational change in P98A mutant Cereblon. In certain embodiments, the method comprises the step of performing a molecular simulation of the structure of the Cereblon.

Accordingly, provided herein are molecular simulations that sample conformational space of the Cereblon protein according to the methods described herein. In certain embodiments, the molecular simulation is a molecular dynamics (MD) simulation.

Molecular simulations can be used to monitor time-dependent processes of the macromolecules and macromolecular complexes disclosed herein, in order to study their structural, dynamic, and thermodynamic properties by solving the equation of motion according to the laws of physics, e.g., the chemical bonds within proteins may be allowed to flex, rotate, bend, or vibrate as allowed by the laws of chemistry and physics. This equation of motion provides information about the time dependence and magnitude of fluctuations in both positions and velocities of the given molecule. Interactions such as electrostatic forces, hydrophobic forces, van der Waals interactions, interactions with solvent and others may also be modeled in MD simulations. The direct output of a MD simulation is a set of “snapshots” (coordinates and velocities) taken at equal time intervals, or sampling intervals. Depending on the desired level of accuracy, the equation of motion to be solved may be the classical (Newtonian) equation of motion, a stochastic equation of motion, a Brownian equation of motion, or even a combination (Becker et al., eds. Computational Biochemistry and Biophysics. New York 2001).

One of ordinary skill in the art will understand that direct output of a MD simulation, that is, the “snapshots” taken at sampling intervals of the MD simulation, will incorporate thermal fluctuations, for example, random deviations of a system from its average state, that occur in a system at equilibrium.

In certain embodiments, the molecular simulation is conducted using the CHARMM (Chemistry at Harvard for Macromolecular Modeling) simulation package (Brooks et al., 2009, “CHARMM: The Biomolecular Simulation Program,” J. Comput. Chem., 30(10):1545-614). In certain embodiments, the molecular simulation is conducted using the NAMD (Not (just) Another Molecular Dynamics program) simulation package (Phillips et al., 2005, “Scalable Molecular Dynamics with NAMD,” J. Comput. Chem., 26, 1781-1802; Kalé et al., 1999, “NAMD2: Greater Scalability for Parallel Molecular Dynamics,” J. Comp. Phys. 151, 283-312). One of skill in the art will understand that multiple packages may be used in combination. Any of the numerous methodologies known in the art to conduct MD simulations may be used in accordance with the methods disclosed herein. The following publications, which are incorporated herein by reference, describe multiple methodologies which may be employed: AMBER (Assisted Model Building with Energy Refinement) (Case et al., 2005, “The Amber Biomolecular Simulation Programs,” J. Comput. Chem. 26, 1668-1688; amber.scripps.edu); CHARMM (Brooks et al., 2009, J. Comput. Chem., 30(10):1545-614; charmm.org); GROMACS (GROningen MAchine for Chemical Simulations) (Van Der Spoel et al., 2005, “GROMACS: Fast, Flexible, and Free,” J. Comput. Chem., 26(16), 1701-18; gromacs.org); GROMOS (GROningen MOlecular Simulation) (Schuler et al., 2001, J. Comput. Chem., 22(11), 1205-1218; igc.ethz.ch/GROMOS/index); LAMMPS (Large-scale Atomic/Molecular Massively Parallel Simulator) (Plimpton et al., 1995, “Fast Parallel Algorithms for Short-Range Molecular Dynamics,” J. Comput. Chem., 117, 1-19; lammps.sandia.gov); and NAMD (Phillips et al., 2005, J. Comput. Chem., 26, 1781-1802; Kale et al., 1999, J. Comp. Phys. 151, 283-312).

Wherein the methods call for a MD simulation, the simulation may be carried out using a simulation package chosen from the group comprising or consisting of AMBER, CHARMM, GROMACS, GROMOS, LAMMPS, and NAMD. In certain embodiments, the simulation package is the CHARMM simulation package. In certain embodiments, the simulation package is the NAMD simulation package.

Wherein the methods call for a MD simulation, the simulation may be of any duration. In certain embodiments, the duration of the MD simulation is greater than 200 ns. In certain embodiments, the duration of the MD simulation is greater than 150 ns. In certain embodiments, the duration of the MD simulation is greater than 100 ns. In certain embodiments, the duration of the MD simulation is greater than 50 ns. In certain embodiments, the duration of the MD simulation of step is about 50, 60, 70, 80, 90, 100, 110, 120, 130, 140, 150, 160, 170, 180, 190, 200, 210, 220, 230, 240, 250, or 250 ns.

In certain embodiments, the molecular simulation incorporates solvent molecules. In certain embodiments, the molecular simulation incorporates implicit or explicit solvent molecules. One of ordinary skill in the art will understand that implicit solvation (also known as continuum solvation) is a method of representing solvent as a continuous medium instead of individual “explicit” solvent molecules most often used in MD simulations and in other applications of molecular mechanics. In certain embodiments, the molecular simulation incorporates water molecules. In certain embodiments, the molecular simulation incorporates implicit or explicit water molecules. In certain embodiments, the molecular simulation incorporates explicit ion molecules.

### 7.2.6 Principal Component Analysis

In certain embodiments, the method optionally comprises the step of principal component analysis (PCA) of the MD trajectory. In certain embodiments, PCA is performed prior to identification of dominant conformations of the Cereblon protein using clustering algorithms (see below). In certain embodiments, PCA is performed using the software AMBER-ptraj (Case et al., 2012, AMBER 12, University of California, San Francisco; Salomon-Ferrer et al., 2013, “An Overview of the Amber Biomolecular Simulation Package,” WIREs Comput. Mol. Sci. 3, 198-210; Amber Home Page. Assisted Model Building with Energy Refinement. Available at: http://ambermd.org, accessed Oct. 26, 2013). PCA reduces the system dimensionality toward a finite set of independent principal components covering the essential dynamics of the system.

### 7.2.7 Calculation of RMSDs

In certain embodiments, the method optionally comprises the step of calculating the root mean square deviation (RMSD) of Ca atoms relative to a reference structure of the Cereblon protein. In certain embodiments, calculation of RMSD is performed to observe the overall behavior of the MD trajectory, prior to identification of dominant conformations of the Cereblon protein using clustering algorithms (see below).

### 7.2.8 Clustering Algorithms

In certain embodiments, the method comprises the steps of using a clustering algorithm to identify dominant conformations of the Cereblon protein from the MD simulation, and selecting the dominant conformations of the protein structure identified from the clustering algorithm.

Clustering algorithms are well known by one of ordinary skill in the art (see, e.g., Shao et al., 2007, “Clustering Molecular Dynamics Trajectories: 1. Characterizing the Performance of Different Clustering Algorithms,” J. Chem. Theory & Computation. 3, 231).

In certain embodiments, 50 or more dominant conformations are selected. In certain embodiments, 100 or more dominant conformations are selected. In certain embodiments, 150 or more dominant conformations are selected. In certain embodiments, 200 or more dominant conformations are selected. In certain embodiments, 250 or more dominant conformations are selected. In certain embodiments, 300 or more dominant conformations are selected.

### 7.2.9 Docking Algorithms

In certain embodiments, the method comprises the step of using a docking algorithm to dock the conformers of the one or more compounds to the dominant conformations of the structure of the Cereblon protein determined from the molecular simulations.

Various docking algorithms are well known to one of ordinary skill in the art. Examples of such algorithms that are readily available include: GLIDE (Friesner et al., 2004 “Glide: A New Approach for Rapid, Accurate Docking and Scoring. 1. Method and Assessment of Docking Accuracy,” J. Med. Chem. 47(7), 1739-49), GOLD (Jones et al., 1995, “Molecular Recognition of Receptor Sites using a Genetic Algorithm with a Description of Desolvation,” J. Mol. Biol., 245, 43), FRED (McGann et al., 2012, “FRED and HYBRID Docking Performance on Standardized Datasets,” Comp. Aid. Mol. Design, 26, 897-906), FlexX (Rarey et al., 1996, “A Fast Flexible Docking Method using an Incremental Construction Algorithm,” J. Mol. Biol., 261, 470), DOCK (Ewing et al., 1997, “Critical Evaluation of Search Algorithms for Automated Molecular Docking and Database Screening,” J. Comput. Chem., 18, 1175-1189), AutoDock (Morris et al., 2009, “Autodock4 and AutoDockTools4: Automated Docking with Selective Receptor Flexiblity,” J. Computational Chemistry, 16, 2785-91), IFREDA (Cavasotto et al., 2004, “Protein Flexibility in Ligand Docking and Virtual Screening to Protein Kinases,” J. Mol. Biol., 337(1), 209-225), and ICM (Abagyan et al., 1994, “ICM-A New Method for Protein Modeling and Design: Application to Docking and Structure Prediction from the Distorted Native Conformation,” J. Comput. Chem., 15, 488-506), among many others.

In certain embodiments, the docking algorithm is DOCK or AutoDock.

### 7.2.10 Identification of Preferred Binding Conformations

In certain embodiments, the method comprises the step of identifying a plurality of preferred binding conformations for each of the combinations compound (ligand) and Cereblon protein (receptor).

In certain embodiments, a clustering algorithm, as described above, is used to identify the preferred binding conformations for each of the combinations of compound and protein. In certain embodiments, the preferred binding conformations are those which have the largest cluster population and the lowest binding energy. In certain embodiments, the preferred binding conformations are the energetically preferred orientation of the compound (ligand) docked to the protein (receptor) to form a stable complex. In certain embodiments, there is only one preferred binding conformation for the docked compound.

### 7.2.11 Optimizing Preferred Binding Conformations

In certain embodiments, the method comprises the step of optimizing the preferred binding conformations using MD, as described above.

In certain embodiments, the MD is scalable MD.

In certain embodiments, the MD uses NAMD software.

### 7.2.12 Calculation of Binding Energies

In certain aspects, provided herein are computational methods for selecting a compound that is likely to induces a conformational change in P98A mutant Cereblon. In certain embodiments, the method comprises the step of calculating binding energies, ΔG_{calc}, for each of the combinations of compound (ligand) and protein (receptor) in the corresponding optimized preferred binding conformations.

Calculation of binding energies using a combination of molecular mechanics and solvation models are well known by one of ordinary skill in the art (see, e.g., Kollman et al., 2000, “Calculating Structures and Free Energies of Complex Molecules: Combining Molecular Mechanics and Continuum Models,” Acc. Chem. Res. 3B, 889-897).

In certain embodiments, the method further comprises outputting the selected calculated binding energies, ΔG_{calc}, and comparing them to physiologically relevant concentrations for each of the combinations of protein and compound. ΔG_{calc} may be compared to ΔG_{obs} for each of the combinations of protein and compound.

### 7.2.13 Modification/Redesign of Compound

In certain embodiments, the method further comprises the step of using a molecular modeling algorithm to chemically modify or design the compound such that it induces a conformational change in P98A mutant Cereblon.

In certain embodiments, the method comprises repeating steps e) through i) for the modified or redesigned compound.

For example, if a chemical moiety of a compound identified as not inducing any conformational change, that chemical moiety may be modified in silico using any one of the molecular modeling algorithms disclosed herein or known to one of ordinary skill in the art. The modified compound may then be retested by repeating steps e) through i) of the methods disclosed herein.

In certain embodiments, the modified or redesigned compound is tested in an in vitro biological assay for inducing a conformational change in P98A mutant Cereblon.

In certain embodiments, the modified or redesigned compound is tested for binding to P98A mutant Cereblon in silico using any of the computational models or screening algorithms disclosed herein.

In certain embodiments, the modified or redesigned compound induces a conformational change in P98A mutant Cereblon.

In certain embodiments, the computational models or screening algorithms disclosed herein for selecting compounds may be combined with any computational models or screening algorithms known to those of ordinary skill in the art for modeling the binding of the compound or modified/redesigned compound to P98A mutant Cereblon and inducing a conformation change in P98A mutant Cereblon.

7.2.14 Selection of New Compound from a Chemical Library

As an alternative to modification/redesign of the compound, a new compound may also be selected from the collections of a chemical or compound library, for example, new drug candidates generated by organic or medicinal chemists as part of a drug discovery program. The new compound may then be tested for inducing a conformational change in P98A mutant Cereblon by repeating steps e) through i) of the methods disclosed herein.

### 7.2.15 Biological Aspects

Optionally, the methods disclosed herein include checking, validating, or confirming the in silico predictions of conformational change in P98A mutant Cereblon with the results of an in vitro biological assay.

Accordingly, in certain aspects, provided herein are biological methods for testing, checking, validating or confirming predictions about a compound's ability to induce a conformational change in P98A mutant Cereblon.

In certain embodiments, the method comprises testing, checking, validating or confirming the predictions regarding the compound or modified compound using standard assaying techniques which are known to those of ordinary skill in the art.

### 8. EXAMPLES

### 8.1 Example 1: Protein Expression and Purification

ZZ-domain-6×His-thrombin-tagged human cereblon (amino acids 5-442) and full-length human DDB1 were co-expressed in SF9 insect cells in ESF921 medium (Expression Systems), in the presence of 50 μM zinc acetate. Cells were resuspended in buffer containing 50 mM Tris-HCl (pH 7.5), 500 mM NaCl, 10 mM imidazole, 10% glycerol, 2 mM TCEP, 1×Protease Inhibitor Cocktail (San Diego Bioscience), and 40,000 U Benzonase (Novagen), and sonicated for 30 s. Lysate was clarified by high speed centrifugation at 108,800 g for 30 min, and clarified lysate was incubated with Ni-NTA affinity resin (Qiagen) for 1 h. Complex was eluted with buffer containing 500 mM imidazole, and the ZZ-domain-6×His tag removed by thrombin cleavage (Enzyme Research) overnight, combined with dialysis in 10 mM imidazole buffer. Cleaved eluate was incubated with Ni-NTA affinity resin (Qiagen), and the flow-through diluted to 200 mM NaCl for further purification over an ANX HiTrap ion exchange column (GE Healthcare). The ANX column was washed with ten column volumes of 50 mM Tris-HCl (pH 7.5), 200 mM NaCl, 3 mM TCEP, followed by ten column volumes of 50 mM Bis-Tris (pH 6.0), 200 mM NaCl, 3 mM TCEP, and the cereblon-DDB1 peak eluted at approximately 200 mM NaCl. This peak was collected and further purified by size-exclusion in buffer containing 10 mM HEPES pH 7.0, 240 mM NaCl, and 3 mM TCEP. Samples were concentrated to 20 mg/mL, aliquoted, flash-frozen, and stored at −80°.

MBP-Ikaros 140-168 and mutants were expressed in E. coli BL21 (DE3) Star cells (Life Technologies) using 2XYT media (Teknova). Cells were induced at OD₆₀₀ 0.6 for 18 h at 16° C. Cells were pelleted, resuspended in buffer containing 200 mM NaCl, 50 mM Tris (pH 7.5), 3 mM TCEP, 10% glycerol, 15 μM zinc acetate, 0.01 mg ml⁻¹ lysozyme (Sigma), 40,000 U benzonase (Novagen), and 1× protease inhibitor cocktail (San Diego Bioscience). Resuspended cells were frozen, thawed for purification, and sonicated for 30 s before high-speed spin at 108,800 g for 30 min. Clarified lysate was incubated with amylose resin (NEB) at 4° C. for 1 h before beads were washed. Protein was eluted with buffer containing 200 mM NaCl, 50 mM Tris (pH 7.5), 3 mM TCEP, 10% glycerol, 150 μM zinc acetate, and 10 mM maltose. Eluate was concentrated and further purified by size exclusion chromatography over a Superdex 200 16/600 column (GE Healthcare) in buffer containing 200 mM NaCl, 50 mM Tris (pH 7.5), 3 mM TCEP, 10% glycerol, and 150 μM zinc acetate.

### 8.2 Example 2: Zinc Finger Protein Purification

Domain boundaries for the individually purified MBP (maltose binding protein)-fused zinc finger domains always include the five amino acids N-terminal and one amino acid C-terminal to the zinc finger domains as named in the text (all numbering from Uniprot Isoform 1). MBP-fused WT and mutant Ikaros, zinc finger domain proteins were expressed in E. coli BL21 (DE3) Star cells (Life Technologies) using 2XYT media (Teknova). Cells were induced at OD600 0.6 for 18 h at 16° C. Cells were pelleted, frozen, thawed for purification, and resuspended in B-PER Bacterial Protein Extraction buffer (Thermo Fisher) containing 15 μM zinc acetate, 40,000 U benzonase (Novagen), and 1× protease inhibitor cocktail (San Diego Bioscience). Lysates were incubated with amylose resin (NEB) at 4° C. for 1 h before beads were washed. Protein was eluted with buffer containing 200 mM NaCl, 50 mM Tris pH 7.5, 3 mM TCEP, 10% glycerol, 15 μM zinc acetate, and 10 mM maltose.

### 8.3 Example 3: Preparation of Samples for Cryo-Electron Microscopy

Frozen 20 mg/mL cereblon-DDB1 complexes were thawed and diluted to 5 mg/mL with buffer containing 20 mM HEPES pH 7, 150 mM NaCl, 3 mM TCEP. Immediately prior to plunge-freezing in liquid ethane, samples are diluted 10-fold in the same buffer supplemented with 0.011% Lauryl Maltose-Neopentyl Glycol (LMNG) to limit complex dissociation. Quantifoil 300 mesh R1.2/1.3 UltrAuFoil Holey Gold Films were plasma cleaned for 7 seconds using a Solarus plasma cleaner (Gatan, Inc.) with a 75% nitrogen, 25% oxygen atmosphere at 15 W. Immediately prior to sample application, grids are further pre-treated with 4 μL solution containing 10 μM cereblon binding-deficient mutant Ikaros residues 140-196 Q146A G151N and blotted from behind with torn Whatman 1 filter paper. 4 μL dilute sample is applied, excess sample was blotted away for 4 s and vitrified by manual plunge freezing into a liquid ethane pool cooled by liquid nitrogen using a manual plunger in a 4° C. cold room >95% humidity. Samples containing IMiDs are prepared the same way, with 10× molar equivalent of drug yielding a buffer concentration 20 mM HEPES pH 7, 150 mM NaCl, 3 mM TCEP, <1% DMSO mixed 2 minutes prior to dilution and application to the grid. Similarly, Ikaros samples are prepared by incubation of 2-fold molar excess Ikaros 2 minutes prior to dilution and application to the grid.

### 8.4 Example 4: Electron Microscopy Data Acquisition

Cryo-EM data were collected on a Thermo-Fisher Talos Arctica transmission electron microscope operating at 200 keV using parallel illumination conditions. Micrographs were acquired using a Gatan K2 Summit direct electron detector, operated in electron counting mode applying a total electron exposure of 62.5 e−/Å. The Leginon data collection software was used to collect 2912 micrographs at 36,000× nominal magnification (1.15 Å/pixel at the specimen level) with a nominal defocus set to −1.5 μm. Variation from the nominally set defocus due to a ˜5% tilt in the stage gave rise to a defocus range (this tilt was not intentional and required service to correct). Stage movement was used to target the center of sixteen 1.2 μm holes for focusing, and an image shift was used to acquire high magnification images in the center of each of the sixteen targeted holes.

### 8.5 Example 5: Hydrogen-Deuterium Exchange (HDX) Detection by Mass Spectrometry (MS)

For peptide identification, differential HDX-MS experiments were conducted as previously described with a few modifications (Chalmers, M. J., et al. (2006). Probing protein ligand interactions by automated hydrogen/deuterium exchange mass spectrometry. Analytical Chemistry, 78(4), 1005-1014). Peptides were identified using tandem MS (MS/MS) with an Orbitrap mass spectrometer (Q Exactive, ThermoFisher). Product ion spectra were acquired in data-dependent mode with the top five most abundant ions selected for the product ion analysis per scan event. The MS/MS data files were submitted to Mascot (Matrix Science) for peptide identification. Peptides included in the HDX analysis peptide set had a MASCOT score greater than 20 and the MS/MS spectra were verified by manual inspection. The MASCOT search was repeated against a decoy (reverse) sequence and ambiguous identifications were ruled out and not included in the HDX peptide set.

For HDX-MS analysis, SAMPLE INFO. Next, 5 μl of sample was diluted into 20 μl D₂O buffer (50 mM Tris-HCl, pH 8; 75 mM KCl; 10 mM MgCl₂) and incubated for various time points (0, 10, 60, 300, and 900 s) at 4° C. The deuterium exchange was then slowed by mixing with 25 μl of cold (4° C.) 0.1M Sodium Phosphate, 50 mM TCEP. Quenched samples were immediately injected into the HDX platform. Upon injection, samples were passed through an immobilized pepsin column (2 mm×2 cm) at 200 μl min⁻¹ and the digested peptides were captured on a 2 mm×1 cm C₈ trap column (Agilent) and desalted. Peptides were separated across a 2.1 mm×5 cm C₁₈ column (1.9 μl Hypersil Gold, ThermoFisher) with a linear gradient of 4%-40% CH₃CN and 0.3% formic acid, over 5 min. Sample handling, protein digestion and peptide separation were conducted at 4° C.

Mass spectrometric data were acquired using an Orbitrap mass spectrometer (Exactive, ThermoFisher). HDX analyses were performed in triplicate, with single preparations of each protein ligand complex. The intensity weighted mean m/z centroid value of each peptide envelope was calculated and subsequently converted into a percentage of deuterium incorporation. This is accomplished determining the observed averages of the undeuterated and fully deuterated spectra and using the conventional formula described elsewhere (Zhang, Z., & Smith, D. L. (1993). Determination of amide hydrogen exchange by mass spectrometry: a new tool for protein structure elucidation. Protein Science, 2(4), 522-531.). Statistical significance for the differential HDX data is determined by an unpaired t-test for each time point, a procedure that is integrated into the HDX Workbench software (Pascal, B. D., et al. (2012). HDX workbench: software for the analysis of H/D exchange MS data. Journal of the American Society for Mass Spectrometry, 23(9), 1512-1521.). Corrections for back-exchange were made on the basis of an estimated 70% deuterium recovery, and accounting for the known 80% deuterium content of the deuterium exchange buffer.

For data rendering, HDX data from all overlapping peptides were consolidated to individual amino acid values using a residue averaging approach. Briefly, for each residue, the deuterium incorporation values and peptide lengths from all overlapping peptides were assembled. A weighting function was applied in which shorter peptides were weighted more heavily and longer peptides were weighted less. Each of the weighted deuterium incorporation values were then averaged to produce a single value for each amino acid. The initial two residues of each peptide, as well as prolines, were omitted from the calculations. This approach is similar to that previously described (Keppel, T. R., & Weis, D. D. (2014). Mapping residual structure in intrinsically disordered proteins at residue resolution using millisecond hydrogen/deuterium exchange and residue averaging. Journal of the American Society for Mass Spectrometry, 26(4), 547-554.). HDX analyses were performed in triplicate, with single preparations of each purified protein/complex. Statistical significance for the differential HDX data is determined by t test for each time point, and is integrated into the HDX Workbench software.

### 8.6 Example 6: In Vitro Ubiquitination Assays

Purified E1, E2, ubiquitin, Cul4A-Rbx1, cereblon-DDB1, and GSPT1 proteins were used to reconstitute the ubiquitination of MBP-fused WT and mutant proteins in vitro. Substrate proteins purified by maltose affinity resin (NEB) were incubated at an approximate concentration of 30 uM. In vitro ubiquitination components described briefly: Human cereblon-DDB1 (cereblon amino acids 40-442 and full length DDB1) was co-expressed in SF9 insect cells and purified by nickel affinity resin (Qiagen), HiTrap ANX column ion exchange (GE Healthcare), and Sephacryl 400 16/60 size-exclusion chromatography (GE healthcare) as described above. Human full-length Cul4A and Rbx1 were co-expressed in SF9 insect cells and purified by nickel affinity resin and Superdex 200 16/60 size-exclusion chromatography. Purified recombinant human Ube1 E1 (E-305), UbcH5a E2 (E2-616), and ubiquitin (U-100H) were purchased from R&D systems. Components were mixed to final concentrations of 10 mM ATP, 1 μM Ube1, 25 μM UbcH5a, 200 μM Ub, 1 μM Cul4-Rbx1, 2 μM Ikaros, and 1 μM cereblon-DDB1, with or without 100 μM compound as indicated in ubiquitination assay buffer (20 mM HEPES pH 7.5, 150 mM NaCl, 10 mM MgCl₂). After pre-incubation of E1, E2, ATP and ubiquitin for 30 min, and separate pre-incubation of MBP-substrate, CRBN-DDB1, Cul4-Rbx1, and compound for 5 min at room temperature, ubiquitination reactions were started by mixing the two pre-incubations. Reactions were incubated at 30° C. for 2 h before separation by SDS-PAGE followed by Coomassie staining or immunoblot analysis.

### 8.7 Example 7: Atomic Model Building and Refinement

Model building and refinement were performed using one round each of morphing and simulated annealing in addition to five real-space refinement macrocycles with atomic displacement parameters, secondary structure restraints, local grid searches, non-crystallographic symmetry, Ramachandran restraints, and global minimization in PHENIX. (Afonine, P. V., et al. (2018). Real-space refinement in PHENIX for cryo-EM and crystallography. Acta Crystallographica Section D: Structural Biology, 74(6), 531-544.) One round of geometry minimization with Ramachandran and rotamer restraints was used to minimize clash scores, followed by a final round of real-space refinement in PHENIX.

UCSF Chimera (Pettersen, E. F., et al. (2004). UCSF Chimera—a visualization system for exploratory research and analysis. Journal of Computational Chemistry, 25(13), 1605-1612.) and ChimeraX (Goddard, T. D., et al. (2018). UCSF ChimeraX: Meeting modern challenges in visualization and analysis. Protein Science, 27(1), 14-25.) were used to interpret the EM reconstructions and atomic models, as well as to generate figures.

### 8.8 Example 8: Open Conformation of DDB1-Bound Cereblon

Cryo-electron microscopy (cryo-EM) was used to examine the population ensemble of DDB1-Cereblon conformers present in solution under various conditions, in order to better understand the mélange of Cereblon conformations. To overcome preferred orientation and partial denaturation/dissociation of the complex during cryo-EM grid preparation, a combination of grid pretreatment with a Cereblon-agnostic substrate, incorporation of mild amphiphilic detergent, and sample dilution immediately prior to plunging improved particle homogeneity and distribution in ice, as described in Example 3.

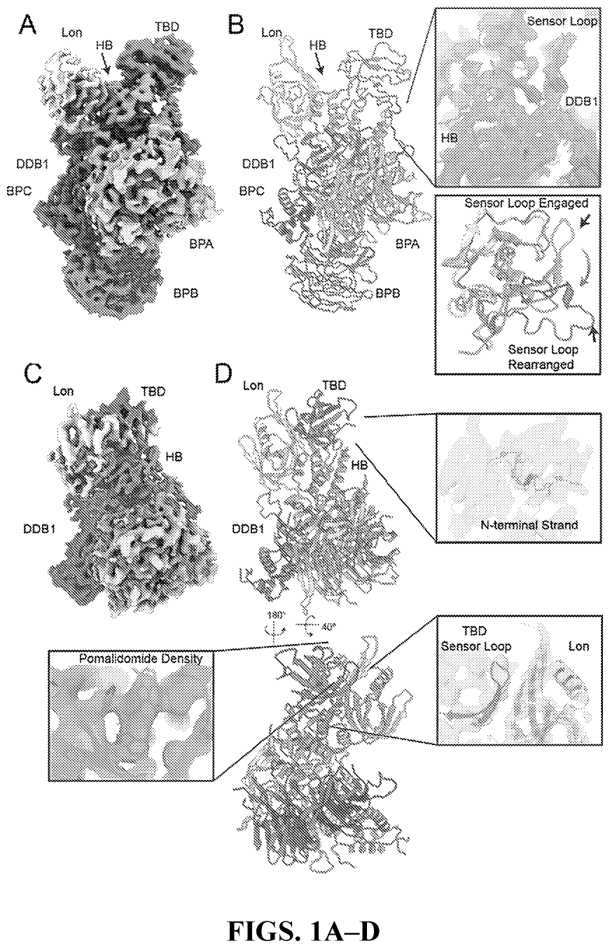

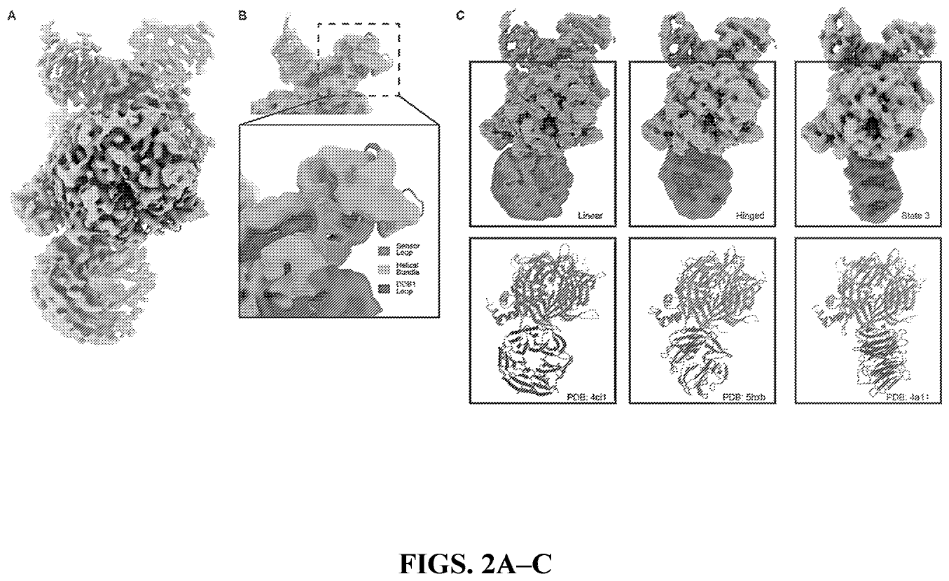

Cereblon co-purified with full-length DDB1 in the apo form was first examined. FIG. 1A depicts a surface representation of an approximately 4 Å resolution cryo-EM reconstruction of Cereblon/DDB1 in the unliganded apo form. The Lon domain is separated from the TBD, while the helical bundle mediates interaction with DDB1, which contains BPA, BPB, and BPC. Similarly, FIG. 2A depicts a 3.5 Å cryo-EM consensus refinement of Cereblon-DDB1 isolated in the apo state. These results are surprisingly because it was found that Cereblon exclusively adopts the open conformation, where the TBD and Lon separated from one another.

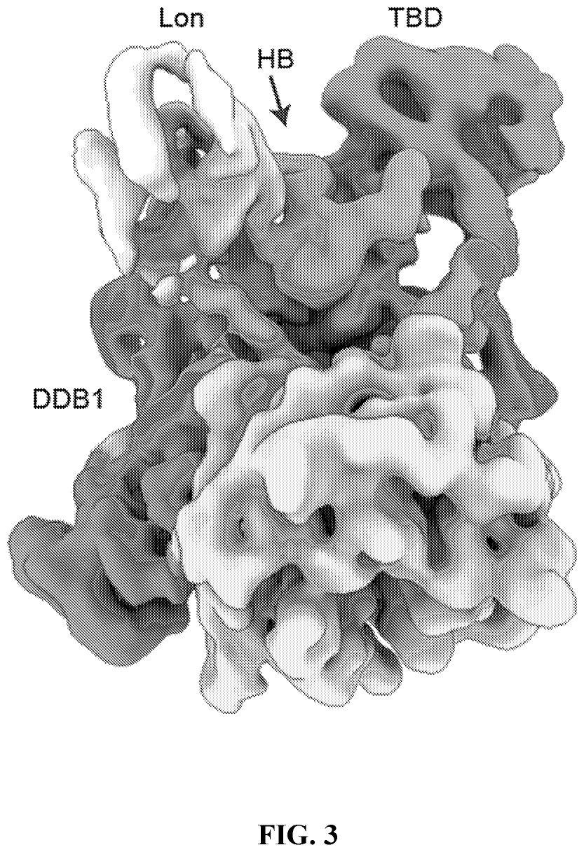

This exclusively open conformer was also observed using DDB1 lacking BPB, Cereblon-DDB1^{ΔBPB}, which is commonly used for X-ray crystallography. FIG. 3 depicts an approximately 10 Å resolution cryo-EM reconstruction of Cereblon-DDB1^{ΔBPB} in the unliganded apo form. Cereblon adopts exclusively the open conformation in absence of drug, using DDB1 lacking the mobile BPB propeller.

These findings confirm the physiological relevance of the open conformer, and these cryo-EM structures provide the first opportunity to characterize this CRBN conformation in the absence of ligand or substrate.

FIG. 1B depicts a ribbon representation of Cereblon-DDB1 protein modelled from density. The upper close-up panel shows details of the sensor loop of Thalidomide Binding Domain (TBD) interacting with the Helical Bundle (HB) and DDB1. The lower close-up panel shows the rearrangement of the sensor loop observed in the apo state relative to the previously observed closed state (PDB: 6BNB). FIG. 2B depicts the same in a Gaussian filtered rendition, highlighting dynamic region of TBD, where the Sensor Loop associates with and a loop in DDB1.

Notably, the sensor loop, a beta-insert hairpin within Cereblon's TBD (residues 346-363) that has been shown to bind directly to IMiDs and substrates in prior structures, is repositioned in the CRBN^{open} conformer. It is observed that the sensor loop disengaged from the TBD fold and instead forming a labile connection with a helix in Cereblon's HB (residues approximately 210-220). Association of the sensor loop in this position between the HB and DDB1 likely contribute to stabilizing the TBD in a single discrete position in the CRBN^{open} conformer. Without being limited by any theory, we posit that substrate recruitment involves a rearrangement of this sensor loop, and that IMiD association with CRBN stabilizes this sensor loop within the TBD to further promote substrate binding.

### 8.9 Example 9: IMiD-Driven Conformational Rearrangement within Cereblon

Saturating amounts of IMiD-type molecules were added to Cereblon-DDB1 for cryo-EM analyses To better understand the drivers of Cereblon closing. Addition of Pomalidomide was sufficient to induce conformational rearrangement within Cereblon for approximately 20% particles. FIG. 1C depicts a surface representation of an approximately 4 Å resolution cryo-EM reconstruction of Cereblon-DDB1 in the closed form in complex with Pomalidomide. The approximately 4 Å resolution structure of CRBN^{closed} shows the approximately 15 kDa TBD positioned adjacent to Lon domain, and unambiguous density within the ligand-binding pocket consistent with Pomalidomide association.

FIG. 1D depicts a ribbon representation of Cereblon-DDB1^{ΔBPB}. The close-up panels detail the N-terminal strand engagement (top right), sensor loop formation (bottom right), and density corresponding to Pomalidomide in CRBN^{closed} (bottom left, Pom shown in rigid-body docked PDB: 6h0g). The sensor loop in CRBN^{closed} is released from the HB tether and adopts the canonical beta-hairpin fold within the TBD, and a portion of the N-terminal strand that is disordered in the CRBN^{open} becomes ordered around the TBD, strengthening the position of the TBD in the closed conformation.

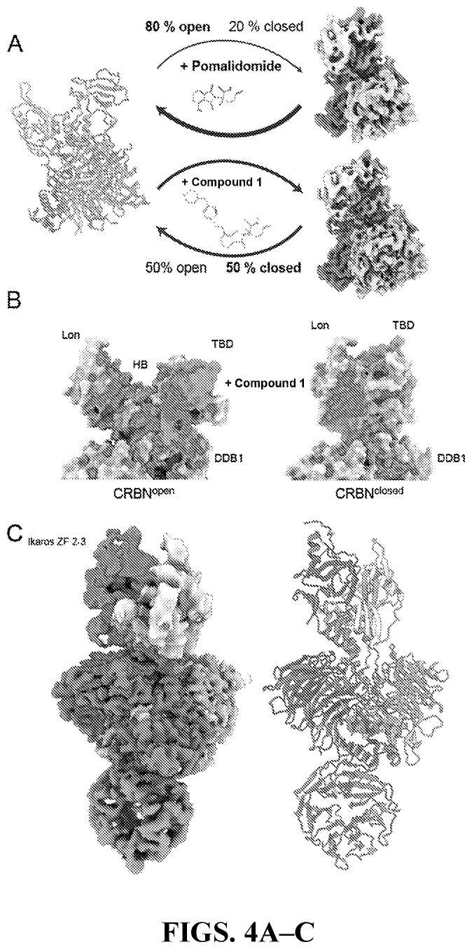

Intriguingly, a minor population of Cereblon was observed to be closed in the presence of Pomalidomide. Without being limited by any theory, we rationalized that next-generation molecules with improved binding affinity may also have a more profound allosteric effect as a contributing factor to improved substrate degradation. Compound 1 is a CELMoD with approximately 20-fold improved affinity. Preparing DDB1-Cereblon in the presence of Compound 1 shifts nearly 50% of particles to the CRBN^{closed} conformer. FIG. 4A depicts how the CRBN^{open} transition to CRBN^{closed} is differentially regulated between Pomalidomide (20% particles adopt CRBN^{closed}) and the CELMoD Compound 1 (50% particles adopt CRBN^{closed}). A surface representation of the approximately 4 Å resolution cryo-EM structure of Pomalidomide-induced CRBN^{closed} is shown in the upper right. On the bottom right is an approximately 3.8 Å resolution cryo-EM density from Compound 1-induced CRBN^{closed}.

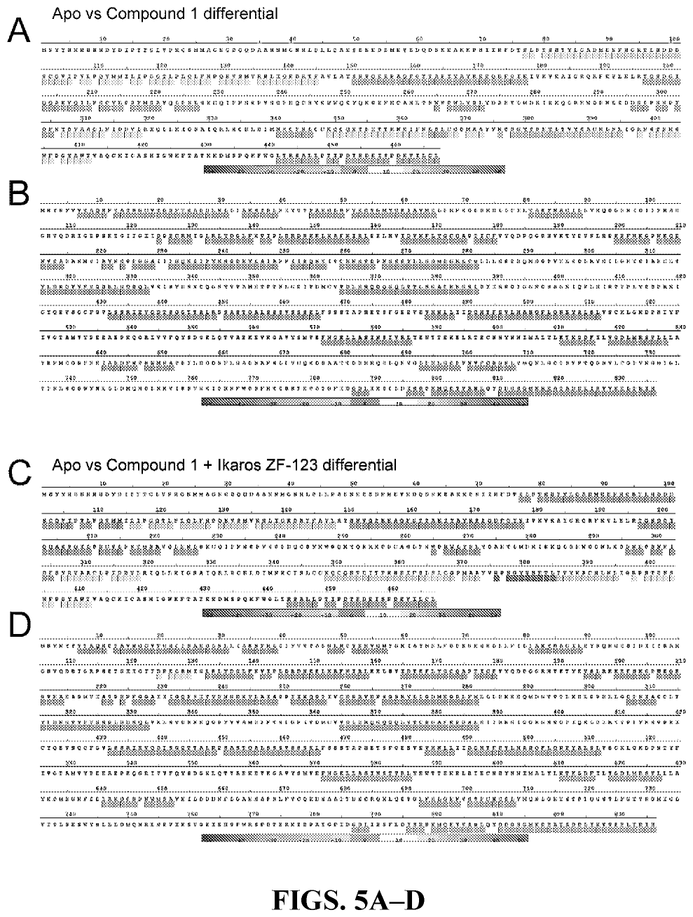

The allosteric influence of IMiD on CRBN closure was further confirmed using hydrogen-deuterium exchange mass spectrometry (HDX-MS) to profile changes in solvent accessibility of DDB1-Cereblon peptides in the presence of Compound 1. FIG. 4B depicts a space-filling representation of Cereblon models with residue-specific shading according to changes in solvency upon addition of Compound 1 as detected by HDX-MS. FIG. 5A depicts a per-residue peptide mapping of Cereblon HDX differential upon addition of Compound 1 to Cereblon-DDB1. FIG. 5B depicts a per-residue peptide mapping of DDB1 HDX differential upon addition of Compound 1 to Cereblon-DDB1. Compared with unliganded DDB1-Cereblon, addition of drug affected solvency for both the sensor loop and residues within the Lon domain and HB, consistent with transition to CRBN^{closed}.

Without being limited by any theory, these results indicate a new modality for drug development, wherein properties of the drug influence not only binding kinetics for Cereblon and neosubstrates, but also the capacity to initiate and maintain the closed conformation as a prerequisite for substrate recruitment.

### 8.10 Example 10: Drug Mediates Recruitment of Ikaros to the Closed Conformation

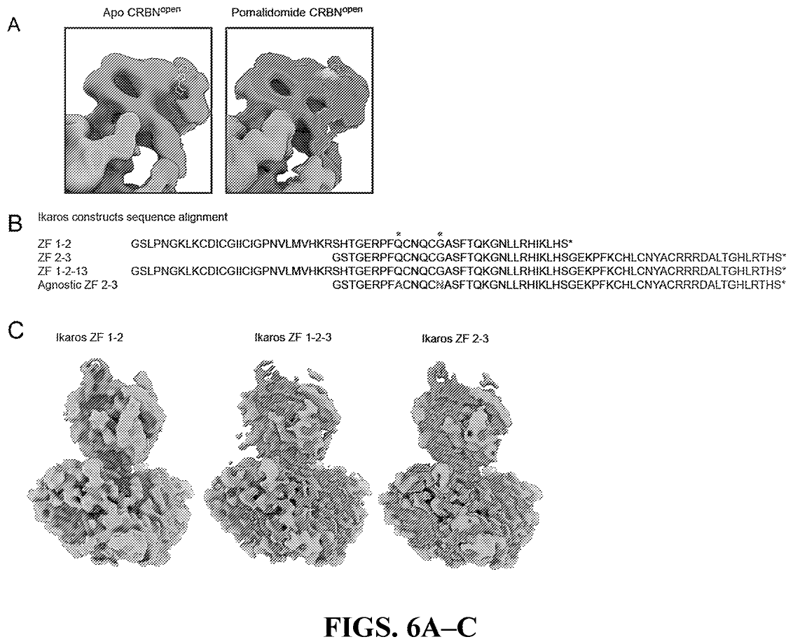

FIG. 6A depicts a zoomed-in view of TBD from low-resolution cryo-EM reconstructions of unliganded apo CRBN^{open} or Pomalidomide-bound CRBN^{open} showing drug is recruited to the open conformation. TBD-Pomalidome from PDB:6h0g is rigid body fit. Comparison of the apo and liganded data reveals that drug also associates with the TBD of CRBN^{open}, suggesting a mechanistic path wherein ligand-binding to the open TBD may promote a rearrangement of the sensor loop prior to TBD closure.

To understand the details of substrate recruitment and positioning within this context, Zinc-finger transcription factor Ikaros was employed, which is the cellular target of Pomalidomide and Compound 1. Constructs containing tandem Zinc-finger domains was generated for increased recruitment efficiency. FIG. 6B depicts sequence alignment for Ikaros proteins used in these studies. Ikaros comprises multiple Zinc-Finger motifs in tandem, here ZF1-2, ZF2-3, ZF1-2-3, and a Cereblon-agnostic mutated ZF2-3 were used for preparation of cryo-EM grids.

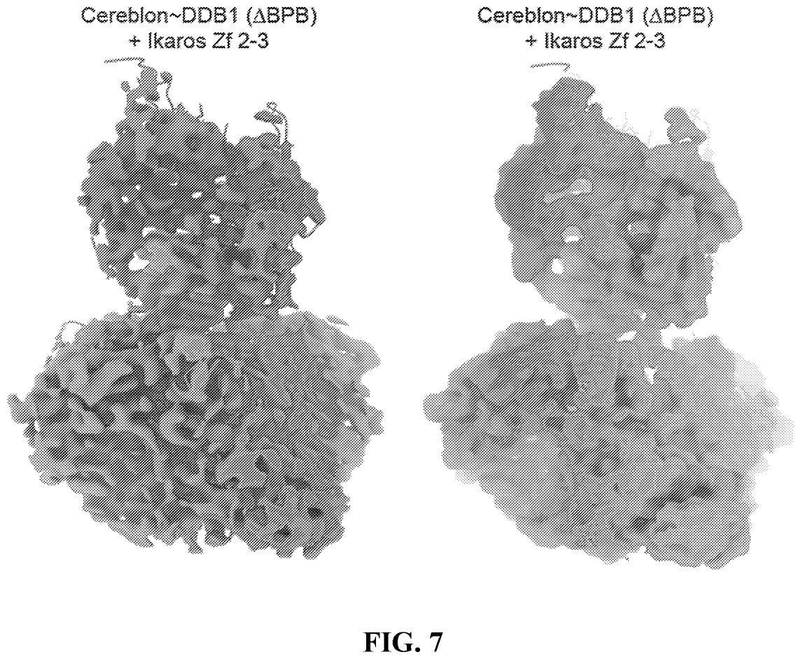

The constructs were incubated individually with DDB1-Cereblon in the presence of Pomalidomide or Compound 1. FIG. 6C depicts an approximately 4 Å cryo-EM reconstructions of Cereblon-DDB1^{ΔBPB} bound to different versions of Ikaros tandem ZF protein. Each of these proteins shows only a single Zinc-finger motif bound to the TBD. Other Zinc-finger domains are not visible, presumably because they are flexibly attached and do not form complex with Cereblon. FIG. 7 depicts 3.4 Å sharpened and unsharpened map of Cereblon-DDB1 bound to Ikaros Zf 2-3. FIG. 4C depicts a composite map of local refinements for Ikaros/Cereblon/DDB1. The tandem, multi-ZF Ikaros 1-2-3 protein is recruited to CRBN^{closed} by Compound 1. Local refinements were performed for DDB1 BPB, DDB1 BPA-BPC with HB, and Cereblon/Ikaros, aligned to consensus refinement and stitched in Chimera. Regardless of drug identity or construct design, single-particle analyses reveal density consistent with the location and positioning of a single Zinc-finger motif in the closed conformation.

FIG. 5C depicts a per-residue peptide mapping of Cereblon HDX differential upon addition of Compound 1 and Ikaros ZF1-2-3 to Cereblon-DDB1. FIG. 5D depicts a per-residue peptide mapping of DDB1 HDX differential upon addition of Compound 1 and Ikaros ZF1-2-3 to Cereblon-DDB1. These results from HDX-MS again confirmed the more profound effects on the residues at this interface. Intriguingly, Ikaros was not detected in the open conformation. Without being limited by any theory, this is consistent with the notion that drug-induced closing occurs prior to substrate recruitment, further underscoring the importance of allosteric regulation in the system.

### 8.11 Example 11: Beta Propeller B of DDB1 Alternates Between Three Distinct Conformations

The position of Ikaros relative to the full CRL4 assembly was investigated in order to gain further insight into the ubiquitination mechanism. Cullin-4 recruits the rigid DDB1-CRBN^{closed}-Ikaros complex by association with the BPB. The distal BPB extends from the DDB1 core as a mobile element, and this flexibility has been implicated in the mechanism of DDB1 as a promiscuous adaptor that bridges Cul4 with structurally diverse DCAF-substrate modules. Correspondingly, BPB has been captured in a variety of conformations in several dozen crystallographic studies.

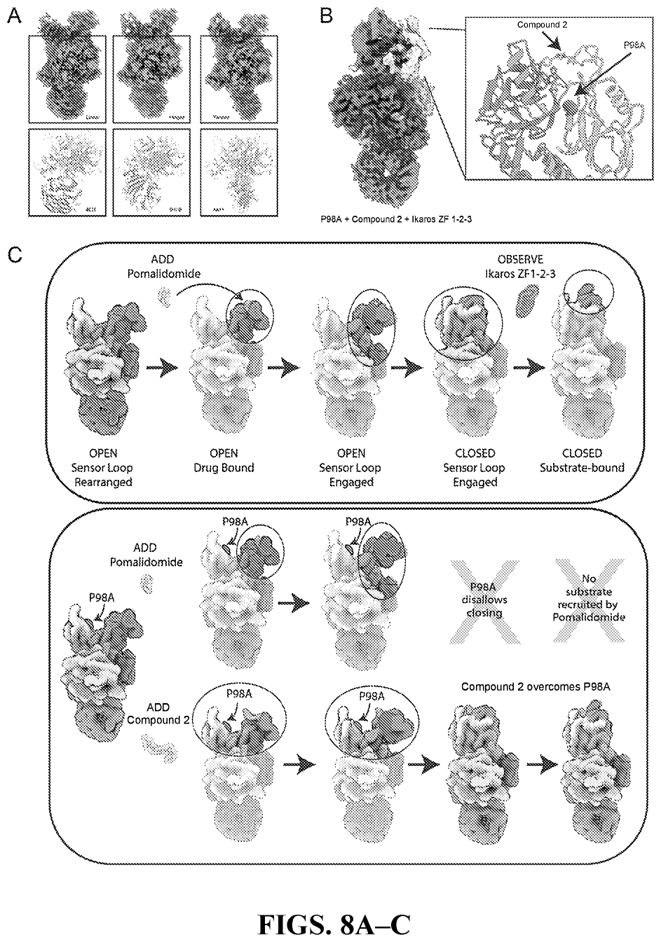

Unliganded Cereblon/DDB1 is described in Example 8. 3D classification of the consensus refinement of unliganded Cereblon/DDB1 yields three positions of DDB1's mobile BPB propeller, which are termed “linear,” “hinged,” and “state 3” (also termed “twisted”). FIG. 8A depicts approximately 3.6 Å resolution cryo-EM reconstructions of Cereblon/DDB1 with BPB in the Linear (top left), Hinged (top middle) or Twisted (top right) positions. These positions of DDB1 BPB are consistent with common orientations observed in crystallography, represented by PDB 4ci1(bottom left), 5hxb (bottom middle), 4a11 (bottom right). FIG. 2C depicts the same cryo-EM and crystallographic models in an alternative rendition.

Image analyses of this region within the apo Cereblon data instead reveals that the BPB adopts three distinct conformations, as opposed to a continuum of motion. In solution, the BPB in approximately 65% of the particles adopts a “linear” orientation, in which the broad face of BPB is in a similar orientation as BPC. An approximately 70° rotated “hinged” orientation, where the BPB face more closely matches BPA is the second major class observed, with approximately 20-30% of particles, and only a minor population contain BPB adopting a third, “twisted” orientation, rotated ˜140° from the linear orientation. Although these distinct orientations were observed in recent low-resolution cryo-EM structures of DDB1-DCAF1 bound to Cullin-4, our structures demonstrate that these BPB positions are intrinsic to DDB1, irrespective of the substrate adaptor module or interaction with Cullin, as depicted in FIG. 8C. Although we were unable to discern allosteric coordination upon ligand- or Ikaros-binding, the population distribution of the states vary significantly compared to those observed for the Cullin-bound DDB1. Notably, the “twisted” orientation that we observe in less than 10% of our particles is the majority conformer when bound to Cullin, suggesting that BPB position may impact the ubiquitination mechanism upon association with Cullin.

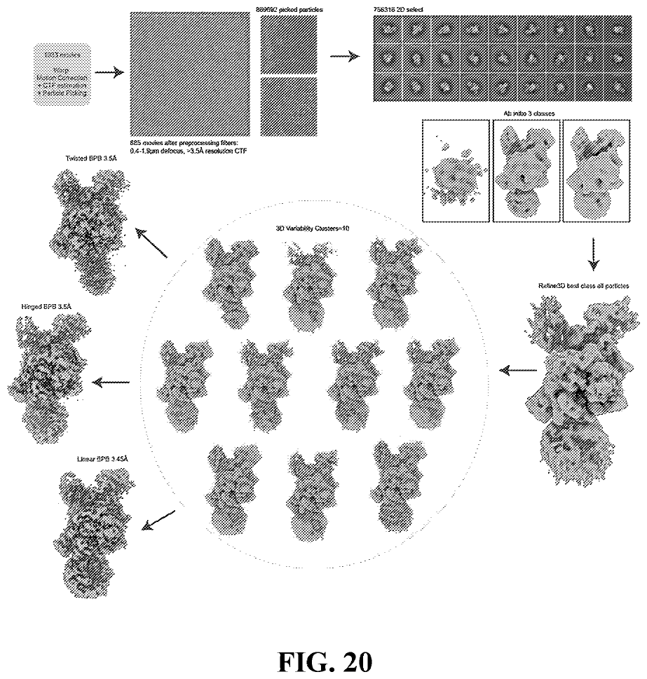

FIG. 20 depicts the typical processing workflow described above for Cereblon/DDB1 in the unliganded apo form. Movies are preprocessed in Warp, particles are imported to CryoSPARC for 2D classification and 3D refinement.

### 8.12 Example 12: Mechanistic Implications for Relapse/Refractory Multiple Myeloma

Given the potential clinical relevance of the observed CRBN allostery, scenarios whereby deficits in this mechanism perturb function was next considered. Patients with mutations distal from the drug-binding site but at locations involved in the open-to-closed transition, such as the interface of TBD and Lon domains, may be refractory to traditional IMiD treatment or susceptible to relapse. One such mutation, P98A within the Lon domain, renders patients unresponsive to Pomalidomide, establishing a need for the development of a new class of CELMoDs. In the case of this mutation, the next-generation CELMoD Compound 2, has been developed for the treatment of relapse/refractory multiple myeloma (RRMM) in a P98A cohort.

To characterize the utility of this class of drugs, we prepared P98A mutant Cereblon as before, saturated with either Pomalidomide or Compound 2 along with Ikaros. Unexpectedly, we find that P98A blocks formation of CRBN^{closed}, yielding exclusively substrate-free open conformers even in the presence of Pomalidomide.

This directly implicates allostery as a mechanistic requirement that is dysregulated in disease and decoupled from drug-binding kinetics. Remarkably, addition of Compound 2 overcomes this mutational challenge and adopts the CRBN^{closed} conformation with Ikaros bound for 25% of particles in the context of P98A,

FIG. 8B depicts an approximately 4 Δ resolution cryo-EM reconstruction of P98A Cereblon/DDB1 complexed with Compound 2 and Ikaros ZF1-2-3. The right panel depicts a ribbon representation of Cereblon highlighting P98 mutation site within the Lon domain, at the interface of TBD in CRBN^{closed}.

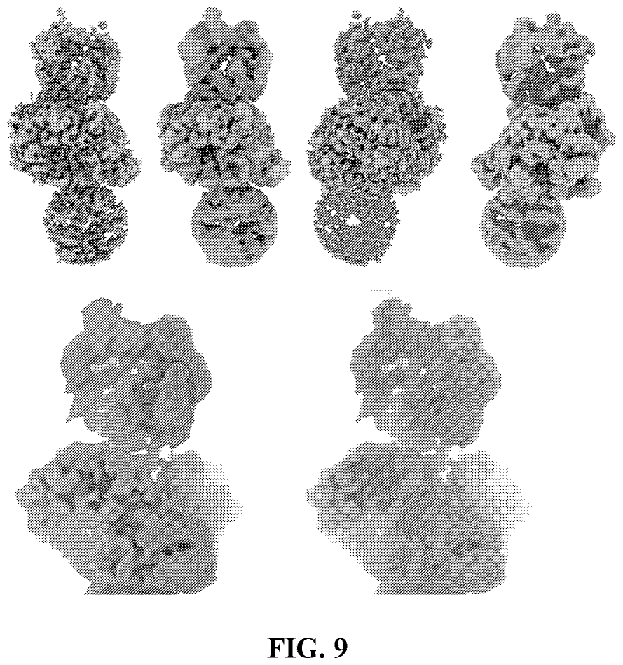

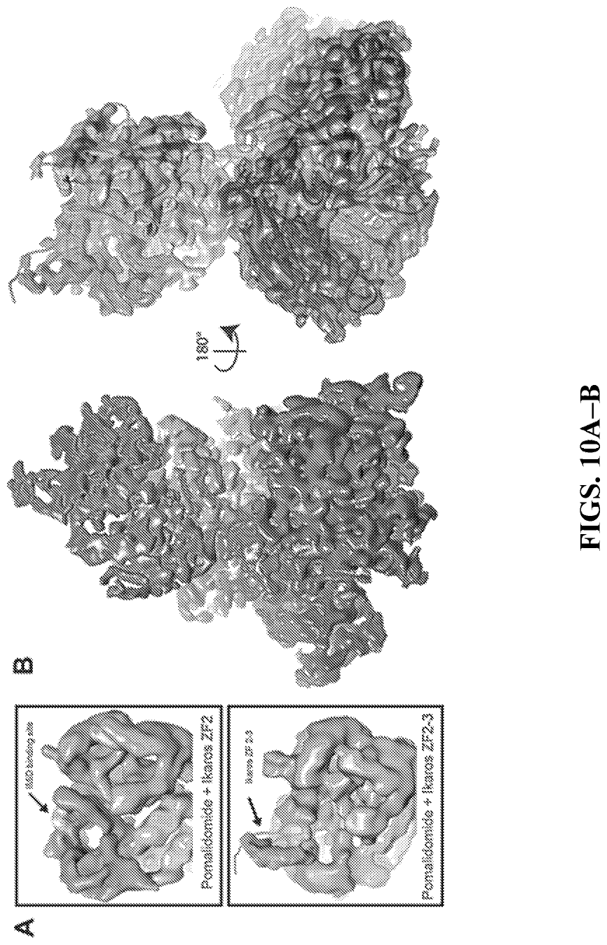

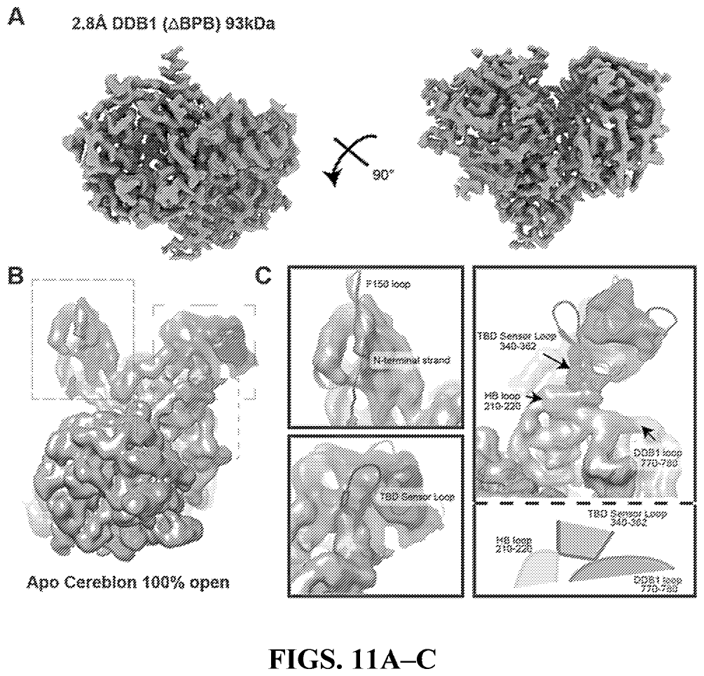

FIG. 9 depicts sharpened and unsharpened maps of Cereblon P98A. The next-generation CELMoD Compound 2 sufficiently recruits Ikaros Zf 2-3 in the context of the P98A mutant, when Pomalidomide is insufficient.

These results highlight the value of chemical control of Cereblon conformation and defining an important mechanism responsible for the success of this new class of CeLMoDs.

### 8.13 Example 13: Open and Closed Conformations of P98A Mutant Cereblon

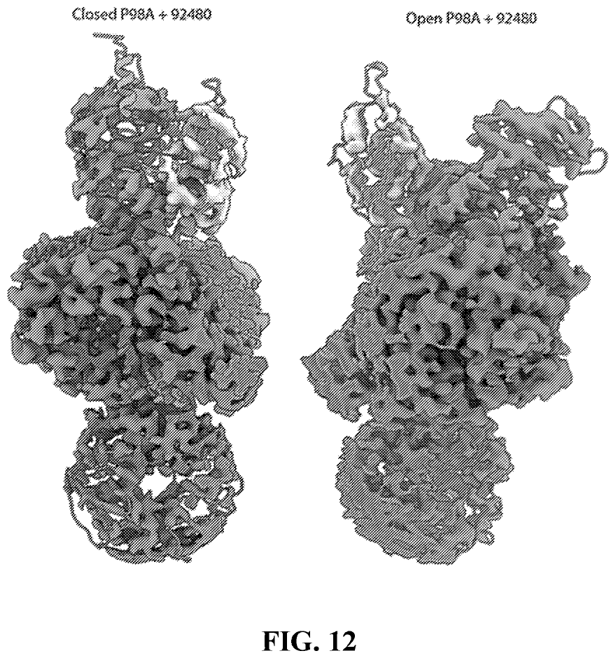

FIG. 12 depicts models of P98A mutant Cereblon in the open and closed conformations, as they best fit the volumes generated from a dataset of P98A mutant Cereblon with Compound 2 added. These models have been refined in PHENIX and improved in Coot/ISOLDE but do not have ligand-bound in these models. The model for the closed conformation also features Ikaros ZF2.

Table 1 below sets forth the atomic coordinates for P98A mutant Cereblon in the open conformation.

Table 2 below sets forth the atomic coordinates for P98A mutant Cereblon in the closed conformation.

Lengthy table referenced here
US20250052749A1-20250213-T00001
Please refer to the end of the specification for access instructions.

Lengthy table referenced here
US20250052749A1-20250213-T00002
Please refer to the end of the specification for access instructions.

### 8.14 Example 14: Computing System

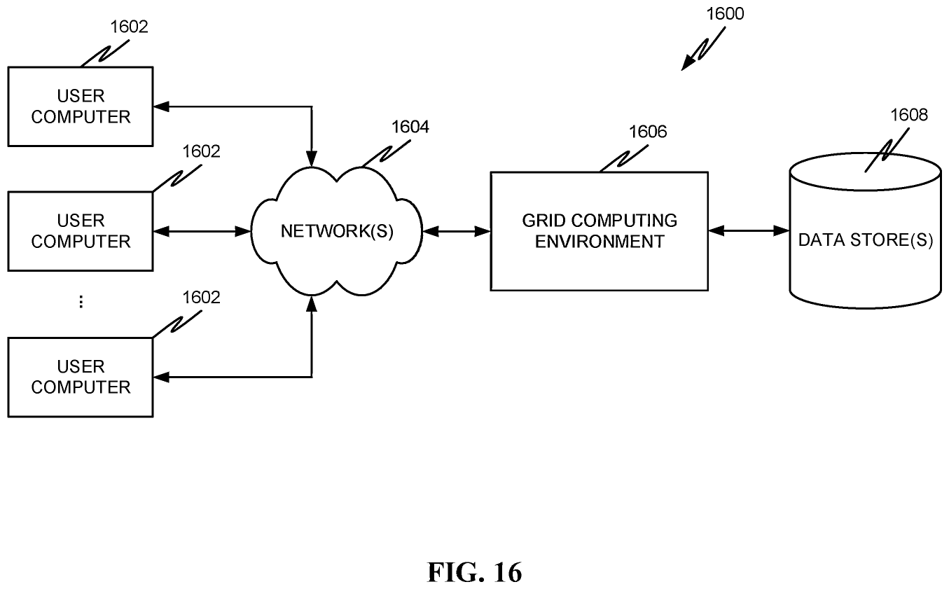

FIG. 16 depicts a grid computing environment for identifying a compound that induces a conformational change in CRBN. As shown in FIG. 16, user computers 1602 can interact with the grid computing environment 1606 through a number of ways, such as over one or more networks 1604. The grid computing environment 1606 can assist users to select a compound that induces a conformational change in CRBN.

One or more data stores 1608 can store the data to be analyzed by the grid computing environment 1606 as well as any intermediate or final data generated by the grid computing environment. However in certain embodiments, the configuration of the grid computing environment 1606 allows its operations to be performed such that intermediate and final data results can be stored solely in volatile memory (e.g., RAM), without a requirement that intermediate or final data results be stored to non-volatile types of memory (e.g., disk).

This can be useful in certain situations, such as when the grid computing environment 1606 receives ad hoc queries from a user and when responses, which are generated by processing large amounts of data, need to be generated on-the-fly. In this non-limiting situation, the grid computing environment 1606 is configured to retain the processed information within the grid memory so that responses can be generated for the user at different levels of detail as well as allow a user to interactively query against this information.

For example, the grid computing environment 1606 receives structural information describing the structure of the CRBN protein, and performs a molecular dynamics simulation of the protein structure. Then, the grid computing environment 1606 uses a clustering algorithm to identify dominant conformations of the protein structure from the molecular dynamics simulation, and select the dominant conformations of the protein structure identified from the clustering algorithm. In addition, the grid computing environment 1606 receives structural information describing conformers of one or more compounds, and uses a docking algorithm to dock the conformers of the one or more compounds to the dominant conformations. The grid computing environment 1606 further identifies a plurality of preferred binding conformations for each of the combinations of protein and compound, and optimizes the preferred binding conformations using molecular dynamics simulations so as to determine whether the compound is able to induce a conformational change in CRBN in the preferred binding conformations.

Specifically, in response to user inquires about whether a compound is able to induce a conformational change in CRBN, the grid computing environment 1606, without an OLAP or relational database environment being required, aggregates protein structural information and compound structural information from the data stores 1608. Then the grid computing environment 1606 uses the received protein structural information to perform molecular dynamics simulations for determining configurations of target protein flexibility (e.g., over a simulation length of greater than 50 ns). The molecular dynamics simulations involve the grid computing environment 1606 determining forces acting on an atom based upon an empirical force field that approximates intramolecular forces, where numerical integration is performed to update positions and velocities of atoms. The grid computing environment 1606 clusters molecular dynamic trajectories formed based upon the updated positions and velocities of the atoms into dominant conformations of the protein, and executes a docking algorithm that uses the compound's structural information in order to dock the compound's conformers to the dominant conformations of the protein. Based on information related to the docked compound's conformers, the grid computing environment 1606 identifies a plurality of preferred binding conformations for each of the combinations of protein and compound. Depending on whether the compound causes a conformational change in CRBN in the preferred binding conformation, the grid computing environment 1606 may redesigns the compound.

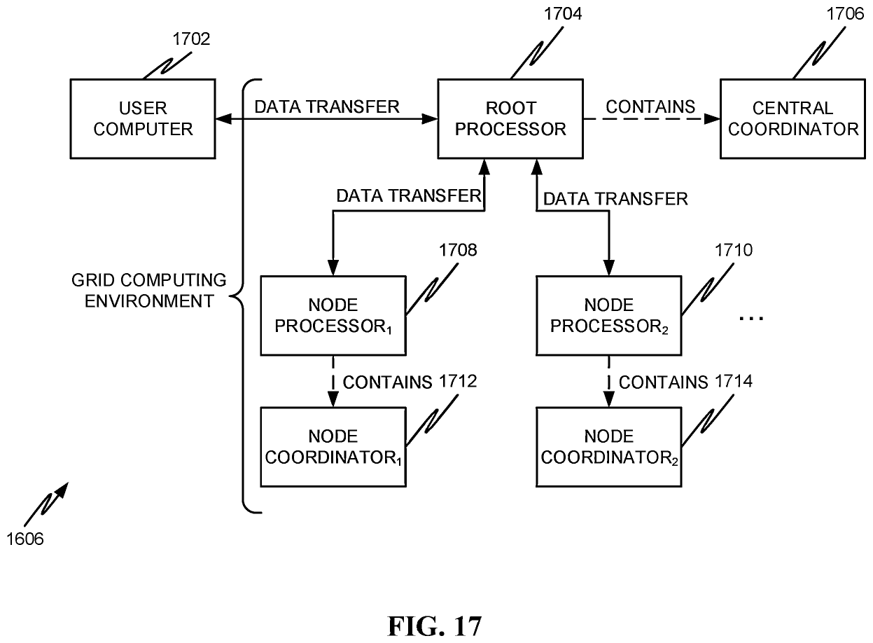

FIG. 17 illustrate hardware and software components for the grid computing environment 1606. As shown in FIG. 17, the grid computing environment 1606 includes a central coordinator software component 1706 which operates on a root data processor 1704. The central coordinator 1706 of the grid computing environment 1606 communicates with a user computer 1602 and with node coordinator software components (1712, 1714) which execute on their own separate data processors (1708, 1710) contained within the grid computing environment 1606.

As an example of an implementation environment, the grid computing environment 1606 can comprise a number of blade servers, and a central coordinator 1706 and the node coordinators (1712, 1714) are associated with their own blade server. In other words, a central coordinator 1706 and the node coordinators (1712, 1714) execute on their own respective blade server. In some embodiments, each blade server contains multiple cores and a thread is associated with and executes on a core belonging to a node processor (e.g., node processor 1708). A network connects each blade server together.

The central coordinator 1706 comprises a node on the grid. For example, there might be 100 nodes, with only 50 nodes specified to be run as node coordinators. The grid computing environment 1306 will run the central coordinator 1706 as a 51st node, and selects the central coordinator node randomly from within the grid. Accordingly, the central coordinator 1706 has the same hardware configuration as a node coordinator.

The central coordinator 1706 may receive information and provide information to a user regarding queries that the user has submitted to the grid. The central coordinator 1706 is also responsible for communicating with the 50 node coordinator nodes, such as by sending those instructions on what to do as well as receiving and processing information from the node coordinators. In one implementation, the central coordinator 1706 is the central point of contact for the client with respect to the grid, and a user never directly communicates with any of the node coordinators.

With respect to data transfers involving the central coordinator 1706, the central coordinator 1706 communicates with the client (or another source) to obtain the input data to be processed. The central coordinator 1706 divides up the input data and sends the correct portion of the input data for routing to the node coordinators. The central coordinator 1706 also may generate random numbers for use by the node coordinators in simulation operations as well as aggregate any processing results from the node coordinators. The central coordinator 1706 manages the node coordinators, and each node coordinator manages the threads which execute on their respective machines.

A node coordinator allocates memory for the threads with which it is associated. Associated threads are those that are in the same physical blade server as the node coordinator. However, it should be understood that other configurations could be used, such as multiple node coordinators being in the same blade server to manage different threads which operate on the server. Similar to a node coordinator managing and controlling operations within a blade server, the central coordinator 1706 manages and controls operations within a chassis.

In certain embodiments, a node processor includes shared memory for use for a node coordinator and its threads. The grid computing environment 1606 is structured to conduct its operations (e.g., matrix operations, etc.) such that as many data transfers as possible occur within a blade server (i.e., between threads via shared memory on their node) versus performing data transfers between threads which operate on different blades. Such data transfers via shared memory are more efficient than a data transfer involving a connection with another blade server.

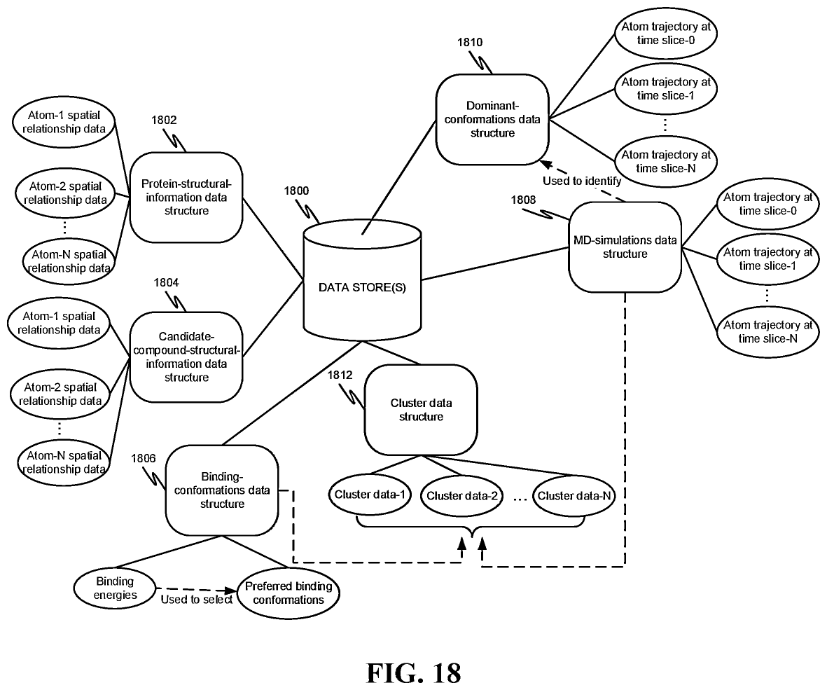

FIG. 18 depicts example schematics of data structures utilized by a compound-selection system. Multiple data structures are stored in a data store 1800, including a protein-structural-information data structure 1802, a candidate-compound-structural-information data structure 1804, a binding-conformations data structure 1806, a molecular-dynamics-simulations data structure 1808, a dominant-conformations data structure 1810, and a cluster data structure 1812. These interrelated data structures can be part of the central coordinator 1706 by aggregating data from individual nodes. However, portions of these data structures can be distributed as needed, so that the individual nodes can store the process data. The data store 1800 can be different types of storage devices and programming constructs (e.g., RAM, ROM, Flash memory, flat files, databases, programming data structures, programming variables, IF-THEN (or similar type) statement constructs, etc.). For example, the data store 1800 can be a single relational database or can be databases residing on a server in a distributed network.

Specifically, the protein-structural-information data structure 1802 is configured to store data related to the structure of the CRBN protein, for example, special relationship data between different atoms. The data related to the structure of the CRBN protein may be obtained from a homology model, an NMR solution structure, an X-ray crystal structure, a molecular model, etc. Molecular dynamics simulations can be performed on data stored in the protein-structural-information data structure 1802. For example, the molecular dynamics simulations involve solving the equation of motion according to the laws of physics, e.g., the chemical bonds within proteins being allowed to flex, rotate, bend, or vibrate. Information about the time dependence and magnitude of fluctuations in both positions and velocities of the given molecule/atoms is obtained from the molecular dynamics simulations. For example, data related to coordinates and velocities of molecules/atoms at equal time intervals or sampling intervals are obtained from the molecular dynamics simulations. Atomistic trajectory data (e.g., at different time slices) are formed based on the positions and velocities of molecules/atoms resulted from the molecular dynamics simulations and stored in the molecular-dynamics-simulations data structure 1808. The molecular dynamics simulations can be of any duration. In certain embodiments, the duration of the molecular dynamics simulation is greater than 50 ns, for example, preferably greater than 200 ns.

Data stored in the molecular-dynamics-simulations data structure 1808 are processed using a clustering algorithm, and associated cluster population data are stored in the cluster data structure 1812. Dominant conformations of the CRBN protein are identified based at least in part on the data stored in the molecular-dynamics-simulations data structure 1808 and the associated cluster population data stored in the cluster data structure 1812. Atomistic trajectory data (e.g., at different time slices) related to the identified dominant conformations are stored in the dominant-conformations data structure 1810.

Data stored in the candidate-compound-structure-information data structure 1804 are processed together with data related to the dominant conformations of the CRBN protein stored in the dominant-conformations data structure 1810. The conformers of the one or more compounds are docked to the dominant conformations of the structure of the CRBN protein using a docking algorithm (e.g., DOCK, AutoDock, etc.), so that data related to various combinations of CRBN protein and compound is determined and stored in the binding-conformations data structure 1806. For example, the compound is an IMiD (e.g. pomalidomide). As an example, the binding-conformations data structure includes data related to binding energies. 2D information of the compound may be translated into a 3D representative structure to be stored in the candidate-compound-structure-information data structure 1804 for docking. Data stored in the binding-conformations data structure 1806 are processed using a clustering algorithm, and associated cluster population data are stored in the cluster data structure 1812. One or more preferred binding conformations are identified based at least in part on the data stored in the binding-conformations data structure 1806 and the associated cluster population data stored in the cluster data structure 1812. For example, the preferred binding conformations include those with a largest cluster population and a lowest binding energy.

The identified preferred binding conformations are optimized using a scalable molecular dynamics simulations (e.g., through a NAMD software, etc.). In certain embodiments, binding energies are calculated (e.g., using salvation models, etc.) for each of the combinations of protein and compound (receptor and ligand) in the corresponding optimized preferred binding conformation(s). The calculated binding energies are output as the predicted binding energies for each of the combinations of protein and compound.

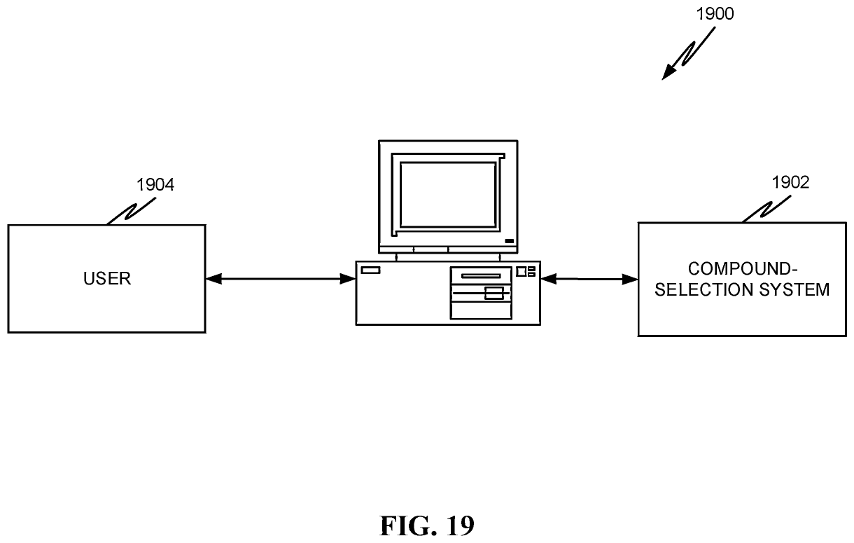

A system can be configured such that a compound-selection system 1902 can be provided on a stand-alone computer for access by a user 1904, such as shown at 1900 in FIG. 19.

Additionally, the methods and systems described herein may be implemented on many different types of processing devices by program code comprising program instructions that are executable by the device processing subsystem. The software program instructions may include source code, object code, machine code, or any other stored data that is operable to cause a processing system to perform the methods and operations described herein. Other implementations may also be used, however, such as firmware or even appropriately designed hardware configured to carry out the methods and systems described herein.

The systems' and methods' data (e.g., associations, mappings, data input, data output, intermediate data results, final data results, etc.) may be stored and implemented in one or more different types of computer-implemented data stores, such as different types of storage devices and programming constructs (e.g., RAM, ROM, Flash memory, flat files, databases, programming data structures, programming variables, IF-THEN (or similar type) statement constructs, etc.). It is noted that data structures describe formats for use in organizing and storing data in databases, programs, memory, or other computer-readable media for use by a computer program.

The systems and methods may be provided on many different types of computer-readable media including computer storage mechanisms (e.g., CD-ROM, diskette, RAM, flash memory, computer's hard drive, etc.) that contain instructions (e.g., software) for use in execution by a processor to perform the methods' operations and implement the systems described herein.

The computer components, software modules, functions, data stores and data structures described herein may be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

The computing system can include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope or of what may be claimed, but rather as descriptions of features specific to particular embodiments. Certain features that are described in this specification in the context or separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments have been described. Other embodiments are within the scope of the following claims. For example, the actions recited in the claims can be performed in a different order and still achieve desirable results.

All publications and patent applications cited in this specification are herein incorporated by reference as if each individual publication or patent application were specifically and individually indicated to be incorporated by reference. Although the foregoing has been described in some detail by way of illustration and example for purposes of clarity of understanding, it will be readily apparent to those of ordinary skill in the art in light of the teachings of the specification that certain changes and modifications may be made thereto without departing from the spirit or scope of the appended claims.

LENGTHY TABLES
The patent application contains a lengthy table section. A copy of the table is available in electronic form from the USPTO web site (https://seqdata.uspto.gov/?pageRequest=docDetail&DocID=US20250052749A1). An electronic copy of the table will also be available from the USPTO upon request and payment of the fee set forth in 37 CFR 1.19(b)(3).

What is claimed:

1. A method of identifying a compound that induces a conformational change in P98A mutant Cereblon (P98A CRBN), wherein the method comprises:
(a) contacting the compound with P98A CRBN; and
(b) assessing the P98A CRBN for a conformational change;
wherein a P98A CRBN conformational change is indicative of a compound that induces the P98A CRBN conformational change.

2. The method of claim 1, wherein the conformational change is a change from an open to closed conformation.

3. The method of claim 2, wherein the C-terminal Thalidomide Binding Domain (TBD) and LON-like domain (LON) domains of the P98A CRBN are in the open conformation.

4. The method of claim 2, wherein the TBD and LON domains are in the closed conformation.

5. The method of claim 1, wherein the conformational change occurs in a cereblon modifying agent (CMA) binding pocket of the P98A CRBN and has an effect on W380, W386 and/or W400; has an effect on E377; or has an effect on V388 of P98A CRBN, and wherein the amino acid numbering correlates to human P98A CRBN.

6. A method of identifying a compound that induces a conformational change in P98A mutant Cereblon (P98A CRBN), wherein the method comprises:
accessing biophysical and dynamics data for P98A CRBN; analyzing the biophysical and dynamics data to identify one or more potential allosteric sites on P98A CRBN;
computationally screening a plurality of chemical compounds to determine a binding energy between each of the subset of allosteric sites and each of the plurality of chemical compounds; computationally modeling the effect of the chemical compounds binding to each of the plurality of allosteric sites; quantifying each of a plurality of conformations of P98A CRBN while bound to the chemical compounds and while not bound to the compounds; ranking the compounds based on achieving a conformation change in P98A CRBN; and selecting a subset of top ranking chemical compounds comprising a potential compound that induced a conformational change in P98A CRBN.

7. The method of claim 6, wherein the biophysical dynamics data is obtained by one or more of nuclear magnetic resonance (NMR), X-ray crystallography, cryogenic electron microscopy (Cryo-EM), neutron scattering, and hydrogen-deuterium exchange.

8. The method of claim 6, wherein the biophysical dynamics data is obtained by Cryo-EM.

9. The method of claim 6, wherein the conformational change is a change from an open to closed conformation.

10. The method of claim 9, wherein the C-terminal Thalidomide Binding Domain (TBD) and LON-like domain (LON) domains of the P98A CRBN are in the open conformation.

11. The method of claim 9, wherein the TBD and LON domains are in the closed conformation.

12. The method of claim 6, wherein the conformational change occurs in a cereblon modifying agent (CMA) binding pocket of the P98A CRBN and has an effect on W380, W386 and/or W400; has an effect on E377; or has an effect on V388 of P98A CRBN, and wherein the amino acid numbering correlates to human P98A CRBN.

13. The method of claim 6, further comprising computationally modeling a derived set of chemical compounds based on strong binding compounds with different functional groups to improve the binding on the allosteric sites.

14. The method of claim 6, wherein the computational modeling is performed using high-throughput computational docking software.

15. A method of identifying a compound that induces a conformational change in P98A mutant Cereblon (P98A CRBN), wherein the method comprises:
(a) using structural information describing the structure of P98A CRBN;
(b) performing a molecular dynamics (MD) simulation of the structure;
(c) using a clustering algorithm to identify dominant conformations of the structure from the MD simulation;
(d) selecting the dominant conformations of the structure identified from the clustering algorithm;
(e) providing structural information describing conformers of one or more compounds;
(f) using a docking algorithm to dock the conformers of the one or more compounds of step (e) to the dominant conformations of step (d);
(g) identifying a plurality of preferred binding conformations for each of the combinations of protein and compound;
(h) optimizing the preferred binding conformations using scalable MD; and
(i) determining if the compound causes a conformation change in P98A CRBN in the preferred binding conformations;
wherein if the compound causes a conformational change in the preferred binding conformations, the compound is identified; or
wherein if the compound does not causes a conformational change in the preferred binding conformations, the compound is not identified; and
wherein said steps (a) through (i) are executed on one or more processors.

16. The method of claim 15, wherein the structural information is obtained by one or more of nuclear magnetic resonance (NMR), X-ray crystallography, cryogenic electron microscopy (Cryo-EM), and a homology model.

17. The method of claim 15, wherein the structural information is obtained by Cryo-EM.

18. The method of claim 15, wherein the conformational change is a change from an open to closed conformation.

19. The method of claim 18, wherein the C-terminal Thalidomide Binding Domain (TBD) and LON-like domain (LON) domains of the P98A CRBN are in the open conformation.

20. The method of claim 18, wherein the TBD and LON domains are in the closed conformation.
